(19) United States
(12) Patent Application Publication
(10) Pub. No.: US 2007/0292895 A1
(43) Pub. Date: Dec. 20, 2007

(54) Assays and methods to detect beta-secretase and its activity in body fluids and tissue extracts

(76) Inventors: Xiao-Ping Shi, Warrington, PA (US); Guoxin Wu, Merion Station, PA (US); Jason Kahana, Schwenksville, PA (US); Adam J. Simon, Yardley, PA (US)

Correspondence Address: MERCK AND CO., INC, P O BOX 2000, RAHWAY, NJ, 07065-0907, US

(21) Appl. No.: 11/803,207

(22) Filed: May 14, 2007

Related U.S. Application Data
(60) Provisional application No. 60/801,965, filed on May 19, 2006.

(51) Int. Cl. G01N 33/573

(52) U.S. Cl. 435/7.4

(57) ABSTRACT

Methods and assays for detecting β-secretase (BACE-1) activity in body fluids or tissue extracts from individuals, particularly individuals who have Alzheimer's disease, are disclosed. A truncated form of BACE-1 was isolated that can be used as a biomarker for Alzheimer's disease.

### CROSS REFERENCE TO RELATED APPLICATIONS

This application claims the benefit of U.S. Provisional Application No. 60/801,965, filed May 19, 2006, the contents of which are incorporated herein by reference in their entirety.

### BACKGROUND OF THE INVENTION

(1) Field of the Invention

The present invention relates to methods and assays for detecting β-secretase (BACE-1) activity in body fluids or tissue extracts from individuals, particularly individuals who have Alzheimer's disease. The present invention further relates to a biomarker for Alzheimer's disease comprising a truncated form of BACE-1.

(2) Description of Related Art

Alzheimer's disease is a neurodegenerative disease of the brain that accounts for the majority of the dementia diagnosed in the elderly. Alzheimer's disease is characterized clinically as a slow and progressive decline in cognitive function, which leaves the end-stage patient dependent on custodial care with death occurring on average about nine years after diagnosis. The pathology of Alzheimer's disease is thought to be a gradual and chronic imbalance between the production and clearance of secreted Aβ peptides resulting in the progressive formation of insoluble amyloid plaques and neurofibrillary tangles. The Aβ peptides, known as Aβ₄₀ and Aβ₄₂, are the result of the proteolytic processing of the amyloid precursor protein (APP) by two enzymes, β- and γ-secretase. Once formed, the Aβ monomers can polymerize and form neurotoxic oligomers that disrupt neuronal function and lead to cell death and memory loss that is the phenotype of Alzheimer's disease (Lacor et al., Neurosci. 24: 10191 (2004)).

β-secretase (BACE-1) cleavage of amyloid precursor protein (APP) is believed to be an essential step in the production of Aβ peptide fragments, which are believed to have a major role in the pathogenesis of Alzheimer's disease (Golde, Brain Pathol. 15: 84 (2005); Beher and Graham, Expert Opin. Investg. Drugs 14: 1385-1409 (2005); Lacor et al., Neurosci. 24:10191 (2004)). Aβ generation is completely abolished in mice deficient for BACE-1 which suggests that BACE-1 is the primary enzyme responsible for cleaving APP at the β-site. BACE-1 is a transmembrane aspartyl protease that cleaves APP at the β-secretase site, SEVKMDAEFR (SEQ ID NO: 1), between the M and D amino acid residues. BACE-1 activity can be measured in both cultured cells (Benjannet et al., J. Biol. Chem. 278: 36264 (2003); Andraut et al., J. Biol. Chem. 278:25859 (2003); Pietrak et al., Anal. Biochem. 342:144-151 (2005)) and various animal tissues, most notably in the brain. Recently, BACE-1 activity was found in cerebral spinal fluid of individuals diagnosed with Alzheimer's disease (Holsinger et al., Annals Neurol. 55:898-899 (2004); Verheijen et al., Clin. Chem. 52 (6): 1168-1174 (2006)) and soluble forms of BACE-1 in CSF has been described (Verheijen et al., ibid.) and in the culture medium of neuroblastoma cell cultures stably expressing BACE-1 (Murayama et al., Biochem. Biophys. Res. Comm. 338:800-807 (2005)).

BACE-1, related secretases and enzymes, and APP processing have been the subject of the following published International Patent Applications:

WO9321526 discloses monitoring the processing of APP in cells by detecting sAPP fragments formed from cleavage of the amino terminus of APP. U.S. Pat. Nos. 5,441,870, 5,604,102, 5,605,811, 5,612,486, 5,721,130, 5,850,003, 6,018,024, 6,245,964 and 6,586,656 have issued from U.S. patent applications based on the international patent application.

WO0023576 discloses a novel BACE-1 with a molecular weight of 61, 81, or 88 kDa, protease complexes comprising the novel BACE-1 and methods for determining the proteolytic activity of the secretase using pure vesicles and APP substrate. U.S. Pat. Nos. 6,245,884, 6,313,268 and 6,627,409 have issued from U.S. patent applications based on the international patent application.

WO03103593 discloses methods for determining the proteolytic activity of in vivo secretases using pure vesicles and APP substrate.

WO0206306 discloses method for identifying agents that modulate activity of the Asp2 (capable of cleaving the β-secretase site) and novel substrates for the Asp2.

WO2005096730 discloses method for determining compounds that bind APP of Aβ using sAPPβ and antibodies specific for sAPPβ.

WO9640885 discloses an isolated purified BACE-1, methods for detecting BACE-1 cleavage of APP, and antibody specific for carboxyl end of sAPPβ derived from cleavage APP having the Swedish mutation. U.S. Pat. Nos. 5,744,346, 5,942,400, 6,221,645, 6,329,163 and 6,852,482 have issued from U.S. patent applications based on the international patent application.

WO9826059 discloses a BACE-1 isolated from 293 cells having an apparent molecular weight of 260-300 kDa when glycosylated.

WO0017369 discloses proteases (Asp2(a) and Asp2(b)) capable of cleaving the β-secretase site of APP. Related international patent applications include WO0123533, which discloses method for assaying human Asp1 α-secretase activity; WO0149097, discloses fragments of Asp2 that are missing the Asp2 transmembrane domain and APP substrates; WO0150829, discloses APP substrates for Asp2; and WO0149098, also discloses APP substrates. U.S. Pat. Nos. 6,825,023, 6,828,117, 6,835,565, 6,844,148, 6,867,018 and 6,913,918 have issued from U.S. patent applications based upon the aforementioned international patent applications.

WO0047618 discloses BACE-1 recombinant cells that produce the enzyme either alone or in combination with some of its natural substrates (β-APPwt and β-APPsw) and methods of selecting compounds that modulate BACE-1. U.S. Pat. No. 6,627,739 has issued from a U.S. patent application based on the international patent application and claims an antibody specific for the BACE-1.

WO03088926 discloses isolated polypeptides (RTN3, RTN4, and rab5c) that can modulate BACE-1 activity.

WO0175435 discloses an isolated y-secretase, a method of detecting cleavage of APP by γ-secretase and detecting cleavage using a pair of fluorescent adducts. U.S. Pat. No. 6,713,248 has issued from a U.S. patent application based on the international patent application.

WO03102177 discloses a modified APP that contains a β-secretase site and a modification that prevents cleavage by α-secretase.

Because BACE-1 appears to be essential for production of Aβ peptide fragments and these Aβ peptide fragments appear to have a major role in the pathogenesis of Alzheimer's disease, methods for detecting BACE-1 activity might provide a means for determining whether an individual has Alzheimer's disease or might be at risk for developing Alzheimer's disease. A method for detecting BACE-1 activity could also be used to monitor Alzheimer's disease in an individual who is undergoing a treatment regime for Alzheimer's disease. A method for detecting Alzheimer's disease would also be useful in drug development in which the effect of drug candidates can be monitored in vivo. While BACE-1 activity can be detected in brain tissue extracts prepared from individuals post mortem, it would be desirable to have a non-invasive method for detecting BACE-1 in biological samples from live individuals. While Verheijen et al., ibid., discloses an indirect method for detecting BACE-1 activity in CSF, the method relies upon an amplification means for detecting BACE-1 activity in CSF. Therefore, there remains a need for methods that are sensitive and specific enough to allow for direct detection of BACE-1 activity. It would be particularly desirable to have a method for detecting BACE-1 activity in serum or plasma.

### BRIEF SUMMARY OF THE INVENTION

The present invention provides methods and assays for detecting β-secretase (BACE-1) activity in body fluids or tissue extracts from individuals, the presence of which can serve as a marker for the presence of Alzheimer's disease in the individuals.

In one aspect, the present invention provides a method for determining whether a biological sample has BACE-1 activity, which comprises incubating the biological sample in a reaction mixture that includes a protease inhibitor that inhibits non-BACE-1 aspartyl proteases but not BACE-1 and a peptide substrate that includes the amino acid sequence NFEV (SEQ ID NO: 3) as a cleavage site for BACE-1 for a time sufficient for any BACE-1 activity in the biological sample to cleave the peptide substrate at the BACE-1 cleavage site to produce a peptide product that has the amino acid sequence NF at the carboxy terminus or EV at the amino terminus; contacting the reaction mixture with an antibody that is specific for the amino acid sequence NF at the carboxy terminus of the peptide product; and detecting the antibody bound to the peptide product, wherein detection of the antibody bound to the peptide product indicates that the sample has BACE-1 activity.

In another aspect, the present invention provides a method for determining whether a biological sample has BACE-1 activity, which comprises providing an antibody that binds BACE-1; incubating the antibody with the biological sample for a time sufficient for the antibody to bind any of the BACE-1 that might be in the biological sample; separating BACE-1 bound to the antibody from the biological sample; incubating BACE-1 bound to the antibody in a reaction mixture that includes a protease inhibitor that inhibits non-BACE-1 aspartyl proteases but not BACE-1 and a peptide substrate of APP that includes the amino acid sequence NFEV (SEQ ID NO: 3) as a cleavage site for BACE-1 for a time sufficient for any of the BACE-1 bound to the antibody to cleave the peptide substrate at the cleavage site for BACE-1 to produce a peptide product that has the amino acid sequence NF at its carboxy terminus or EV at its amino terminus; contacting the reaction mixture with an antibody that is specific for the amino acid sequence NF at the carboxy terminus of the peptide product; and detecting the antibody bound to the peptide product, wherein detection of the antibody bound to the peptide product indicates that the biological sample has BACE-1 activity.

The method can be performed using biological samples such as tissue extracts, particularly brain tissue extracts, cerebral spinal fluid (CSF) or extracts prepared from CSF, plasma or serum or extracts prepared from plasma, serum, or whole blood. The method can also be performed with purified or partially purified BACE-1 samples prepared from tissues, CSF or plasma or serum.

In particular aspects of the method, the protease inhibitor that inhibits non-BACE-1 aspartyl proteases but not BACE-1 is pepstatin A. The reaction is performed at an acidic pH, preferably at a pH of about 4.5. Optionally, the reaction mixture further includes one or more additional protease inhibitors to such proteases as chymotrypsin, thermolysin, papain, pronase, pancreatic extract and trypsin.

In further aspects of the method, the peptide substrate comprises the amino acid sequence EVNFEVEF (SEQ ID NO: 7). Further still, the peptide substrate comprises an amino acid sequence selected from the group consisting of KTEEISEVNFEVEFR (SEQ ID NO: 8) and REVNFEVEFR (SEQ ID NO: 9).

Because BACE-1 activity has been associated with Alzheimer's disease, the methods and kits of the present invention are useful for determining whether an individual has Alzheimer's disease and other forms of amyloid beta associated dementias, including Mild Cognitive Impairment; whether an individual is at risk of developing Alzheimer's disease; whether a drug candidate is efficacious for treating Alzheimer's disease; and whether a treatment regime for an individual who has Alzheimer's disease is efficacious.

Thus, in one aspect the present invention provides a method for detecting Alzheimer's disease in an individual, which comprises obtaining a biological fluid sample from the individual and detecting a peptide product in the form of a truncated BACE-1 having an apparent molecular weight of about 56 kDa in the biological fluid, wherein the presence of said peptide product indicates the individual has Alzheimer's disease.

Thus, in another aspect the present invention provides a method for determining whether an individual is at risk of developing Alzheimer's disease in an individual, which comprises obtaining a biological fluid sample from the individual and detecting a peptide product in the form of a truncated BACE-1 having an apparent molecular weight of about 56 kDa in the biological fluid, wherein the presence of said peptide product indicates the individual has or is at risk for developing Alzheimer's disease.

In another aspect the present invention provides a method for determining the efficacy of a treatment for Alzheimer's disease in an individual, which comprises obtaining a biological fluid sample from the individual before treatment and at various times during the treatment; detecting a peptide product having an apparent molecular weight of about 56 kDa in the biological fluid samples; and, comparing the amount of said peptide product in the samples obtained at various times during treatment to the amount in the sample before treatment to determine the efficacy of the treatment.

In a further aspect, the present invention provides a method for determining the efficacy of a drug candidate for treating Alzheimer's disease, which comprises obtaining biological fluid samples from an individual before treatment of the individual with the drug candidate and at various times during treatment; detecting a peptide product having an apparent molecular weight of about 56 kDa in the biological fluid samples and comparing the amount of said in samples obtained at various times during treatment to the amount in the sample before treatment to determine the efficacy of the drug candidate for treating Alzheimer's disease.

The present invention further provides a biomarker for Alzheimer's disease comprising a carboxy terminal truncated BACE-1, which has an apparent molecular weight of about 56 kDa. The biomarker can used as a biomarker on its own for Alzheimer's disease, as a component of a multi-analyte panel comprising other markers for Alzheimer's disease, or in conjunction with the methods for detecting BACE-1 activity in biological fluids disclosed herein. In another aspect, said biomarker comprises a 50% inhibitory concentration (IC₅₀) value for a BACE-1 inhibitor obtained from a biological sample from an individual which is greater than the IC₅₀ value for the BACE-1 inhibitor from a biological sample from an individual that does not have Alzheimer's disease.

The present invention further provides a kit for an assay for determining whether a biological sample has β-secretase (BACE-1) activity, which comprises at least one protease inhibitor that inhibits non-BACE-1 aspartyl proteases; a peptide substrate that includes the amino acid sequence NFEV (SEQ ID NO:3) as a cleavage site for the BACE-1; an antibody that is specific for an amino acid sequence NF at the carboxy terminus or EV at the amino terminus of the peptide product produced when the peptide substrate is cleaved by the BACE-1; and instructions for performing the assay. Optionally, the kit can further include a BACE-1 specific inhibitor, a recombinant BACE-1, or both.

In further aspects of the kit, the peptide substrate comprises the amino acid sequence EVNFEVEF (SEQ ID NO: 7). Further still, the peptide substrate comprises an amino acid sequence selected from the group consisting of KTEEISEVNFEVEFR (SEQ ID NO: 8) and REVNFEVEFR (SEQ ID NO: 9). In various aspects of the kit, the peptide substrate is labeled at the amino terminus, for example, the peptide substrate is labeled at the amino terminus with biotin.

### BRIEF DESCRIPTION OF THE DRAWINGS

FIG. 1 shows a Western blot of MES membrane preparation from WT and BACE-1 knockout (KO) mice. A BACE-1 protein band was detected using the JHU070604 anti-BACE-1 antibody at 1:1000 fold dilution in the pellet of WT mice, but was not present in KO mice.

FIG. 2A shows the relative BACE-1 expression levels in wild-type (WT), heterozygote, and homozygote BACE-1 KO mice. α-tubulin levels are shown as gel loading controls.

FIG. 2B shows mouse brain BACE-1 activity of wild-type (WT), heterozygote, and homozygote BACE-1 KO mice.

FIG. 3A shows a typical HPLC trace showing the peaks observed in the presence of REVNFEVEFR (SEQ ID NO:8) peptide substrate labeled at the amino terminus with coumarin-only. BACE-1-specific activity was measured in the presence of 10 μM pepstatin A.

FIG. 3B shows a typical HPLC trace showing the peaks observed in the presence of brain extract with substrate and protease inhibitors but without pepstatin A. BACE-1-specific activity was measured in the presence of 10 μM pepstatin A.

FIG. 3C shows a typical HPLC trace showing the peaks observed in the presence of brain extract with substrate, protease inhibitors, and 10 μM pepstatin A. BACE-1-specific activity was measured in the presence of 10 μM pepstatin A.

FIG. 3D shows a typical HPLC trace showing the peaks observed in the presence of brain extract with substrate, protease inhibitors, 10 μM pepstatin A, and 10 μM statine-val (a peptide BACE-1 inhibitor). BACE-1-specific activity was measured in the presence of 10 μM pepstatin A.

FIG. 3E shows a typical HPLC trace showing the peaks observed in the presence of brain extract with substrate, protease inhibitors, 10 μM pepstatin A, and 10 μM Merck-3 (Stachel et al., J. Med. Chem. 47: 6447-50 (2004)). BACE-1-specific activity was measured in the presence of 10 μM pepstatin A.

FIG. 4 shows brain BACE-1 activity measured as the area of product peak. Pi represents protease inhibitor, PepA represents Pepstatin A. E64 is a cysteine protease inhibitor, Stat (V) represents statine-Val.

FIG. 5A shows a dose-dependent titration of brain BACE-1 activity by BACE-1 specific inhibitor Merck-3.

FIG. 5B shows a dose-dependent titration of brain BACE-1 activity by Merck-3 using Baculo-expressed BACE-1.

FIG. 6 shows a Baculo-BACE-1 standard curve, which shows titration of BACE-1 enzymatic activity with the amount of Baculo-BACE-1 concentration and enables quantification of endogenous enzyme activity against a recombinant standard.

FIG. 7 shows a titration of CSF BACE-1 activity as a function of the amount of CSF volume used in the reaction mixture with substrate.

FIG. 8 shows a titration of CSF BACE-1 activity as a function of the duration of incubation of substrate with CSF.

FIG. 9 shows CSF BACE-1 activity in the presence of protease inhibitors (PI), non-specific aspartyl protease, pepstatin A (10 μM), heat inactivation, and a BACE-1 specific inhibitor, Merck-3 (10 μM).

FIG. 10 shows relative BACE-1 activity in a two-fold serial dilution of human and rhesus CSF samples.

FIG. 11A shows dose-dependent inhibition of human CSF BACE-1 enzymatic activity with a BACE-1 specific inhibitor, Merck-3.

FIG. 11B shows CSF BACE-1 activity in autopsy confirmed Alzheimer's subjects (N=27) compared to age-matched controls (N=29). The decreased activity observed in AD subjects compared to controls are statistically meaningful in this analysis when adjusted for age of each subject (p<0.03).

FIG. 12 shows a titration of plasma BACE-1 activity as a function of anti-BACE-1 antibody concentration. There is a concentration dependent and a saturated increase in plasma BACE-1 activity dependent on anti-BACE-1 antibody concentration.

FIG. 13 shows a titration of plasma BACE-1 activity as a function of plasma volume. BACE-1-specific activity increases with plasma volume and is completely blocked by specific BACE-1 inhibitors, Statine-Val and Merck-3, at a 10 μM concentration.

FIG. 14 shows that BACE-1 activity in plasma is pH dependent with the highest level of BACE-1 activity at an acidic pH of about 4.5.

FIG. 15 shows dose-dependent inhibition of plasma BACE-1 activity by Merck-3. Merck-3 has an IC₅₀ of about 50 nM in the presence of protease inhibitors (PI) and pepstatin A.

FIG. 16 shows BACE-1 activity in extracts prepared from rhesus monkey CSF after immunocapture with an amino terminal epitope specific antibody EE-17 and no activity in extracts after immunocapture with carboxy terminal capture epitope specific antibodies 5832 or LK-16. Membrane bound full length BACE-1 (mBACE) is isolated from a cell line expressing human BACE-1; BBACE (aa460) is a carboxy terminal truncated BACE-1 consisting of amino acids 1 to 460; Rabbit IgG (rIgG) is a negative control.

FIG. 17 shows a Western blot of rhesus monkey CSF extract after immunoprecipitation of BACE-1 on a Statine-Val column. After immunoprecipitation of 10 mL rhesus monkey CSF, a truncated BACE-1 species with a molecular weight of about 56 kDa was evident. Membrane bound full length BACE-1 (mBACE); carboxyl terminal truncated BACE-1 consisting of amino acids 1 to 460 (BBACE).

FIG. 18A shows mass spectrometry data using LC-MSMS on a Thermo-Electron LTQ and search results confirming the identity of the band identified in the Western blot of FIG. 17 as BACE-1. Three peptides from a tryptic digest of rhesus BACE-1 were unambiguously derived from BACE-1.

FIG. 18B shows the parent peptide and m/z data for a tryptic peptide from amino acid positions 58 to 68 of BACE-1 and consisting of amino acid sequence GSFVEMVDNLR (SEQ ID NO:13).

FIG. 18C shows the parent peptide and m/z data for a tryptic peptide from amino acid positions 86 to 94 of BACE-1 and consisting of amino acid sequence SIVDSGTTN (SEQ ID NO:15).

FIG. 18D shows the parent peptide and m/z data for a tryptic peptide from amino acid positions 67 to 75 of BACE-1 and consisting of amino acid sequence VEINGQDLK (SEQ ID NO:14).

FIG. 18E shows the experimental scheme for confirming the identity of the carboxy terminal truncated BACE-1 by LC-MSMS with LTQ.

### DETAILED DESCRIPTION OF THE INVENTION

Definitions

The term “BACE-1” refers to β-secretase (See, e.g., Vassar et al., Science 286: 735-741 (1999)), Asp-1, Memapsin-2 BACE, BACE-1, beta-secretase, beta-site APP-cleaving enzyme. As used herein, the term BACE-1 is taken to include all mammalian forms of the naturally occurring enzyme(s) with ability to cleave APP. The term as used herein also includes all recombinant forms, mutations, and other variants of such enzyme so long as these maintain a functional capability to catalyze the cleavage of APP at the appropriate cleavage site KMDA in the wild type APP sequence.

The term “individual” refers to any mammal, including but not limited to humans, monkeys, apes, dogs, rabbits, guinea pigs, and rodents.

The term “Alzheimer's disease” as used herein also refers to early forms of cognitive dysfunction including, but not limited to, “Mild Cognitive Impairment” or “MCI”.

The present invention provides a novel and sensitive method for detecting or determining β-secretase (BACE-1) activity in biological samples. The method can detect BACE-1 activity not only in tissue extracts and cerebral spinal fluid (CSF), but also in plasma or serum. Thus, the method of the present invention provides a non-invasive assay for detecting BACE-1 activity. The method is useful for monitoring the effect of drug candidates for treating Alzheimer's disease (AD) in vivo. The method is also useful for diagnosing Alzheimer's disease in an individual, for monitoring the consequence of a course of treatment for Alzheimer's disease in an individual, or for determining whether an individual is at risk of developing Alzheimer's disease.

The method and the BACE-1 activity it measures as described herein can be used as a biomarker for Alzheimer's disease. The method is also useful when it is combined with a multi-analyte biomarker panel. As a non-limiting illustrative example, the combination of BACE activity, Aβ42, Tau and pTau provides a composite panel that meets the NIA criteria for sensitivity and specificity for biomarkers for use in classifying AD (Regan Research Institute and NIA Consensus Report of the working group on ‘Molecular and Biochemical Markers of Alzheimer's Disease,’ reported at Neurobiology of Aging, 19(2): 109-116 (1998)). Other combinations including BACE activity with other APP catabolites and Tau species are disclosed and claimed in U.S. Ser. Nos. 60/801962 and 60/900,396 and which is incorporated by reference in its entirety.

Using the method of the present invention, Applicants have established that BACE-1 is present and active in bodily fluids such as CSF, plasma or serum. The presence of BACE-1 activity in CSF has been corroborated by Verheijen et al., Clin. Chem. 52(6): 1168-1174 (2006), who reported detecting BACE-1 activity and a soluble form of BACE-1 in CSF. Applicants have also found a unique, truncated form of BACE-1 in bodily fluids, having an apparent molecular weight of 56 kDa. The truncated form of BACE-1 identified by Applicants may also be useful as a biomarker for Alzheimer's disease.

In its basic form, the present invention provides a two-step assay that enables the direct detection of BACE-1 enzymatic activity in biological samples in body fluids such as serum, plasma or cerebral spinal fluid (CSF) or in tissue, such as brain, or in extracts prepared from any one of the aforementioned biological samples. In the first step, a biological sample is obtained from an individual and contacted with a mixture comprising a peptide substrate having a BACE-1 cleavage site that has been modified to be cleaved by BACE-1 at a rate that is faster than the rate exhibited by the wild-type or the Swedish mutation substrate when cleaved by BACE-1. In general, the reaction mixture is at a pH of between about 4.0 and 5.0. Preferably the reaction mixture is at a pH of about 4.5. The reaction mixture is incubated under conditions sufficient for any BACE-1 that might be in the biological sample to cleave the peptide substrate to produce two peptide fragments: an amino terminal peptide product and a carboxy terminal peptide product. In the second step, the amino terminal peptide product is detected using an antibody that is specific for the neo-epitope at the carboxy terminus of the peptide product created upon BACE-1 cleavage. Alternatively, the carboxy terminal peptide product is detected using an antibody that is specific for the neo-epitope formed at the amino terminus of the peptide product created upon BACE-1 cleavage. Detecting the amino terminal peptide product or carboxy terminal peptide product in the reaction mixture indicates that the biological sample has BACE-1 activity. Conversely, the absence of the amino-terminal peptide product or the carboxy terminal peptide product indicates that the biological sample does not have BACE-1 activity.

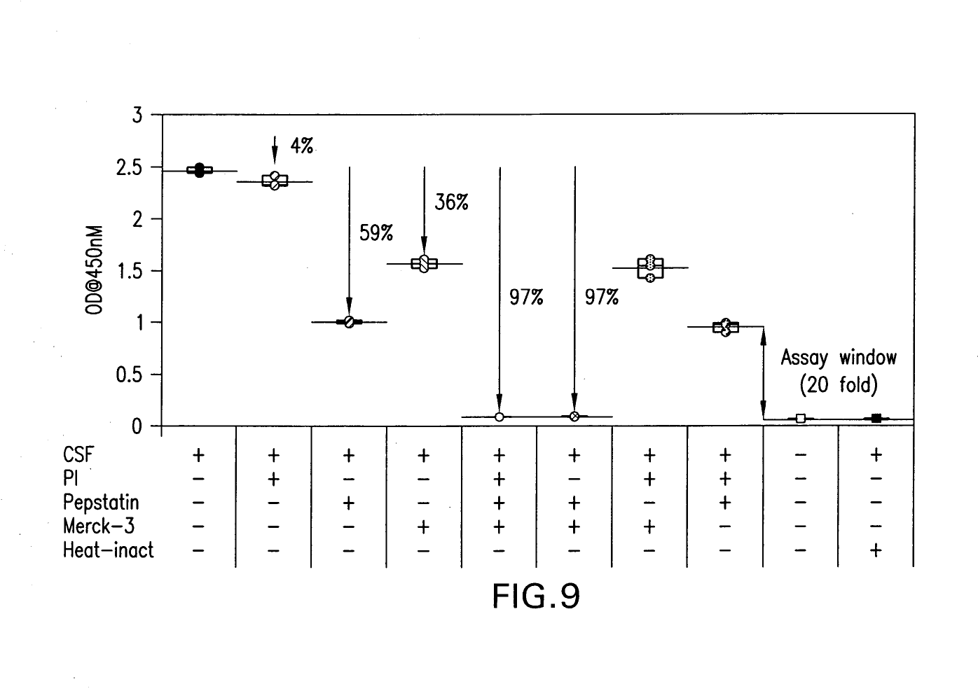

To avoid cleavage of the peptide by endogenous aspartyl proteases that might be present in the biological sample, the reaction mixture further includes an aspartyl protease inhibitor that does not have BACE-1 inhibitory activity. For example, pepstatin A is an aspartyl protease inhibitor that does not inhibit BACE-1 activity. Optionally, the reaction mixture can further include one or more additional protease inhibitors such as to proteases like chymotrypsin, thermolysin, papain, pronase, pancreatic extract, and trypsin. The inclusion of at least one protease inhibitor that inhibits aspartyl proteases but not BACE-1 to the reaction mixture is an important component of the method because it enhances both specificity and sensitivity of the method. Specificity is enhanced because as shown in Example 2, FIG. 9, non-specific aspartyl protease activity, which accounted for about 59% of the protease activity in CSF, could be abolished by adding the aspartyl protease inhibitor, Pepstatin A, to the reaction. The remaining activity was shown to be essentially only BACE-1 activity because the activity could be completely abolished with a BACE-1-specific inhibitor or by heat inactivation. Thus, use of an aspartyl protease inhibitor enhances specificity such that essentially the only aspartyl protease activity remaining in the reaction mixture is BACE-1 activity. FIG. 9 further shows that the BACE-1-specific activity in CSF has a 20-fold window.

In particular aspects, prior to performing the first step reaction, an extract of the biological sample is prepared by mixing the sample with an antibody specific for BACE-1 to separate any BACE-1 that might be in the sample from the other components or contaminants of the sample. In this way, BACE-1 can be purified from components or contaminants in the sample that might be able to fully or partially inhibit BACE-1 activity. Separating BACE-1 from other components or contaminants of the biological sample can also provide a sample that is enriched for BACE-1. Once BACE-1 is separated from the other components or contaminants of the sample, it can either be eluted from the antibodies and used in the first step or BACE-1 bound to the antibodies is used directly in the first step. In a particularly useful embodiment, the antibodies specific for BACE-1 are bound to the walls of a reaction or test tube or the wells of a multi-well plate; the sample is contacted to the bound antibodies; the sample is removed and the bound antibodies and any BACE-1 bound to the antibodies are then contacted with the above reaction mixture containing the peptide substrate as described herein. Example 3 provides such an example where serum samples were incubated with antibodies specific for BACE-1 bound to the surface of wells coated with protein A to capture any BACE-1 that might be present in the samples.

An important aspect of the present invention is using a peptide substrate that has a BACE-1 cleavage site (β-site) that has been modified to be cleaved by BACE-1 at a rate that is faster than the rate the wild-type or Swedish mutation cleavage site is cleaved by BACE-1, which further increases the sensitivity of the method. Therefore, rather than using a peptide that comprises the wild-type KMDA (SEQ.ID.NO:1) amino acid sequence at the β-site or the Swedish mutation NLDA (SEQ.ID.NO:2) amino acid sequence β-site, the peptide substrate has a BACE-1 cleavage site or β-site that comprises an amino acid sequence that is cleaved by BACE-1 at a rate greater than the rate BACE-1 cleaves either the wild-type or Swedish BACE-1 cleavage sites.

The Swedish mutation is an APP molecule having a mutant β-site that has been described in Mullan et al., Nature Genet. 1: 345 (1992) and Citron et al., Nature 360: 672 (1992). It has been shown that cultured cells that express a cDNA encoding APP bearing the Swedish version of the BACE-1 cleavage site produce about six- to eight-fold more Aβ than cells expressing wild-type APP (Citron et al., Nature 360: 672-674 (1992)). Genetically engineered peptide substrates for BACE-1 with mutated β-sites have been disclosed in International Patent Application Nos. WO2002094985 and WO2004099376 and in corresponding U.S. Patent Application Publication Nos. 20030200555 and 20050032190, now U.S. Pat. Nos. 7,196,163 and 7,132,401, respectively, and are incorporated herein by reference as if set forth at length. International Patent Application WO02094985 provides in Table 2, 256 sequences predicted to be good BACE-1 substrates. Mutant β-site amino acid sequences that are cleaved by BACE-1 at a greater rate than the wild type or Swedish cleavage sites include, but are not limited to, amino acid sequences NFAA (SEQ ID NO:4), NFEA (SEQ ID NO:5); NFAV (SEQ ID NO:6), and NFEV (SEQ ID NO:3). However, peptides having the NFEV (SEQ ID NO:3) sequence for the β-secretase cleavage site (β-site) are believed to be particularly good substrates for BACE-1. In particular, an APP molecule having a modified BACE-1 cleavage site comprising the amino acid sequence NFEV (SEQ. ID. NO:3) is claimed as such a substrate (U.S. Pat. No. 7,196,163). Shi et al. in J. Alzheimer's Disease 7: 139-148 (2005) describe a mutant APP genetically engineered to comprise the NFEV (SEQ ID NO:3) amino acid sequence at the β-site. This mutation enhanced the cleavage rate by BACE-1 of the mutant APP by 100-fold relative to the wild-type substrate or 10-fold relative to the Swedish mutation variant. Furthermore, as disclosed in WO02094985, the peptide having the amino acid sequence EVNFEVEF (SEQ ID NO:7) at the β-site was cleaved by BACE-1 at a rate about 60-fold greater that the rate of cleavage of the Swedish cleavage site. Therefore, substrates having the amino acid sequence NFEV (SEQ ID NO: 3) or EVNFEVEF (SEQ ID NO:7) comprising the BACE-1 cleavage site are preferred.

BACE-1 cleaves peptide substrates comprising at least the amino acid sequence NFEV (SEQ ID NO:3) between the F and E amino acid residues to produce two peptide fragments: an amino terminal peptide product having the amino acid sequence NF at the carboxy terminus and a carboxy terminal peptide product having the amino acid sequence EV at the amino terminus. In the second step, the amino terminal peptide product is detected using an antibody that is specific for the NF neo-epitope that was created at the carboxy terminus of the peptide upon BACE-1 cleavage. Alternatively, the carboxy terminal peptide product is detected using an antibody that is specific for the EV neo-epitope created at the amino terminus of the peptide upon BACE-1 cleavage. Detecting the amino terminal peptide product or carboxy terminal peptide product in the reaction mixture indicates that the biological sample has BACE-1 activity. Conversely, the absence of the amino-terminal peptide product or the carboxy terminal peptide product indicates that the biological sample does not have BACE-1 activity.

In particular aspects of the present invention, the peptide substrate is an 8-mer, 10-mer, or 15-mer. In further embodiments, the peptide substrate is a 10-mer that has the amino acid sequence REVNFEVEFR (SEQ ID NO: 8) or a 15-mer that has the amino acid sequence KTEEISEVNFEVEFR (SEQ ID NO: 9). In further embodiments, the substrate can be a proenzyme that includes any one of the amino acid sequences disclosed herein and which requires cleavage by BACE-1 for activation of the enzyme. For example, the proenzyme substrate disclosed in Verheijen et al., ibid, can have a BACE-1 cleavage site that comprises any one of the amino acid sequences disclosed herein.

Novel elements of the present invention include but are not limited to using a peptide substrate with a BACE-1 cleavage site having high sensitivity to BACE-1 cleavage to directly measure BACE-1 enzymatic activity, for example, the peptides disclosed herein having the NFEV (SEQ ID NO:3) amino acid sequence comprising the BACE-1 cleavage site and adding to the reaction mixture and preferably to the biological fluid or extract thereof, at least one aspartyl protease inhibitor that inhibits aspartyl proteases but does not inhibit BACE-1, for example, the aspartyl protease inhibitor pepstatin A and optionally, further including in the reaction mixture at least one or more additional protease inhibitors to proteases such as chymotrypsin, thermolysin, papain, pronase, pancreatic extract, and trypsin.

Other novel elements which can be included in the present invention include (1) performing the method with aliquots of the biological sample in the presence of a BACE-1 specific inhibitor, for example, Statine-Val or Merck-3 (Compound 3 in Stachel et al, J. Med. Chem. 47: 6447-50 (2004)), to confirm that any detected protease activity in the biological fluid or tissue that produced the amino terminal peptide product with the neo-epitope at the carboxy terminus and the carboxy terminal peptide product with the neo-epitope at the amino terminus was due to BACE-1 activity in the biological fluid and not due to activity of some other protease; (2) using a recombinant BACE-1 standard, for example, baculovirus-produced BACE-1, to quantify relative BACE-1 activity on a standard curve; (3) detecting BACE-1 activity in blood products (plasma, serum); and(4) using an NFEV substrate which enables direct high sensitivity measure of enzyme activity

In preferred aspects, the peptide substrate is labeled at the amino terminus for detection of the amino terminal peptide product with an NF amino acid sequence at the carboxy terminus (NF neo-epitope) produced upon cleavage of the peptide substrate by BACE-1. The peptide substrate can be labeled at the amino terminus with a fluorophore or labeled with receptor ligand. For example, the peptide substrate can be labeled with the fluorophore coumarin. Upon cleavage of the peptide substrate with BACE-1, the amino terminal peptide product labeled with coumarin at the amino terminus is detected using a chromatographic method or an antibody to capture the peptide product. Alternatively, the peptide substrate is labeled at the amino terminus with the receptor ligand biotin. Upon cleavage of the peptide substrate with BACE-1, the amino terminus peptide product labeled with biotin at the amino terminus is captured with streptavidin followed by detection of the captured peptide product with an antibody or aptamer specific for the NF amino acid sequence at the carboxy terminus of the peptide product that was formed upon cleavage of the peptide substrate with BACE-1. The NF neo-epitope specific antibody or aptamer can be directly or indirectly labeled with a fluorphore, horseradish peroxidase, alkaline phosphatase, europium, ruthenium, or other label which enables detection or the NF neo-epitope specific antibody can be detected using a second antibody that is specific for the NF neo-epitope specific antibody. The second antibody is labeled with a fluorphore, horseradish peroxidase, alkaline phosphatase, europium, ruthenium, or other label which enables detection.

In an alternative embodiment, the peptide substrate is labeled at the carboxy terminus for detection of the carboxy terminal peptide product with the EV amino acid sequence at the amino terminus (EV neo-epitope) produced upon cleavage of the peptide substrate with BACE-1. The peptide substrate can be labeled at the carboxy terminus with a fluorophore or labeled with receptor ligand. For example, the peptide substrate can be labeled with the fluorophore coumarin. Upon cleavage of the peptide substrate with BACE-1, the carboxy terminal peptide product labeled with coumarin at the carboxy terminus is detected using a chromatographic method or an antibody to capture the peptide product. Alternatively, the peptide substrate is labeled at the carboxy terminus with the receptor ligand biotin. Upon cleavage of the peptide substrate with BACE-1, the carboxy terminus peptide product labeled with biotin at the carboxy terminus is captured with streptavidin followed by detection of the captured peptide product with an antibody or aptamer specific for the EV amino acid sequence at the amino terminus of the peptide product that was formed upon cleavage of the peptide substrate with BACE-1. The EV neo-epitope specific antibody or aptamer can be directly or indirectly labeled with a fluorophore, horseradish peroxidase, alkaline phosphatase, europium, ruthenium, or other label which enables detection or the EV neo-epitope specific antibody can be detected using a second antibody that is specific for the EV neo-epitope specific antibody. The second antibody is labeled with a fluorophore, horseradish peroxidase, alkaline phosphatase, europium, ruthenium, or other label which enables detection.

In a further aspect, the labeled peptide is detected using a chromatographic method such as HPLC instead of antibodies specific for the NF or EV neo-epitope. Therefore, instead of contacting the reaction mixture with an antibody that is specific for the amino acid sequence NF at the carboxy terminus of the peptide product and detecting the antibody bound to the peptide product, the reaction mixture is directly assays on an HPLC gradient and the amount of fluorescence corresponding to position in the gradient where the peptide is expected to elute from the HPLC is determined wherein an increase in fluorescence indicates BACE-1 activity. In a further still aspect, the labeled peptide is labeled at both ends with a fluorescence donor-accepter or donor-quencher pair and cleavage is detected by measuring the ratio of fluorescence of the donor fluorophore to the fluorescence of the accepter fluorophore wherein a change in the ratio indicates BACE-1 activity.

The above assays can be performed in various formats, for example, IGEN's ECL-based technology, HTRF, Alpha Screen technology, and other technologies known to those of ordinary skill in the art.

In particular aspects of the assays, the peptide substrate can be labeled with a fluorescence resonance energy transfer (FRET) donor-accepter pair or donor-quencher pair. If a sample has BACE-1 activity, the peptide substrate is cleaved and the ratio of donor fluorescence to accepter fluorescence changes over time during the reaction. If the ratio does not change over time during the course of the reaction, then the sample does not have BACE-1 activity. For example, the peptide substrate can be labeled with a fluorescence donor such as coumarin at the amino terminus and a fluorescence accepter such as fluorescein at the carboxy terminus. If the peptide substrate is cleaved, the fluorescein fluorescence signal decreases and the coumarin fluorescence signal increases strongly. Therefore, if the sample contains BACE-1 activity, there is an increase in the ratio of donor/accepter fluorescence over time during the course of the reaction. If there is no increase in the ratio over time during the course of the reaction, then the sample does not contain BACE-1 activity. If the accepter or quencher is in very close proximity to the donor, fluorescence of the donor is completely quenched. Upon cleavage of the substrate with BACE-1, the quencher is no longer in close proximity and a fluorescence signal is emitted from the donor which can be detected.

The antibodies used in the method disclosed herein are either specific for the neo epitope comprising the terminal NF or EV amino acid residues, which are produced when the peptide substrate is cleaved by BACE-1 between the F and E amino acid residues of NFEV (SEQ ID NO:3), or specific for the BACE-1 enzyme. The term “antibodies” is intended to be a generic term which includes polyclonal antibodies, monoclonal antibodies, Fab fragments, single V_{H} chain antibodies such as those derived from a library of camel or llama antibodies or camelized antibodies (Nuttall et al., Curr. Pharm. Biotechnol. 1: 253-263 (2000); Muyldermans, J. Biotechnol. 74: 277-302 (2001)), and recombinant antibodies. The term “recombinant antibodies” is intended to be a generic term which includes single polypeptide chains comprising the polypeptide sequence of a whole heavy chain antibody or only the amino terminal variable domain of the single heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides) (see, Schmiedl et al., J. Immunol. Meth. 242: 101-114 (2000); Schultz et al., Cancer Res. 60: 6663-6669 (2000); Dübel et al., J. Immunol. Meth. 178: 201-209 (1995); and in U.S. Pat. No. 6,207,804 B1 to Huston et al.). Construction of recombinant single V_{H} chain or scFv polypeptides which are specific against an analyte can be obtained using currently available molecular techniques such as phage display (de Haard et al., J. Biol. Chem. 274: 18218-18230 (1999); Saviranta et al., Bioconjugate 9: 725-735 (1999); de Greeff et al., Infect. Immun. 68: 3949-3955 (2000)) or polypeptide synthesis. In further embodiments, the recombinant antibodies include modifications such as polypeptides having particular amino acid residues or ligands or labels such as horseradish peroxidase, alkaline phosphatase, fluors, and the like. Further still embodiments include fusion polypeptides which comprise the above polypeptides fused to a second polypeptide such as a polypeptide comprising protein A or protein G.

The antibodies specific for NF or EV neo-epitopes can be produced by methods known in the art. For example, polyclonal and monoclonal antibodies can be produced by methods as described in Harlow and Lane, Antibodies: A Laboratory Manual. Cold Spring Harbor Laboratory Press: Cold Spring Harbor, N.Y. (1988). Altered antibodies such as chimeric, humanized, camelized, CDR-grafted, or bifunctional antibodies can also be produced by methods well known in the art. Such antibodies can also be produced by hybridoma, chemical synthesis or recombinant methods described, for example, in Sambrook et al., supra, and Harlow and Lane, supra. Both anti-peptide and anti-fusion protein antibodies can be used, for example, Bahouth et al., Trends Pharmacol. Sci. 12: 338 (1991); Ausubel et al., Current Protocols in Molecular Biology (John Wiley and Sons, N.Y. (1989). WO2004099376 discloses preparation of antibodies specific for the NF neo-epitope formed upon cleavage of the NFEV (SEQ ID NO:3) amino acid sequence of the peptide substrate. Antibodies specific for BACE-1 can be purchased commercially.

Peptide aptamers that specifically bind to either the NF or EV neo-epitopes generated by BACE-1 cleavage of the NFEV (SEQ ID NO:3) sequence comprising the BACE-1 cleavage site of the peptide substrate can be prepared and used in an assay in which these novel epitopes are generated, for example, Hoppe-Seyler and Butz, J. Mol. Med. 78: 426-430 (2000). Methods for identifying peptide aptamers that specifically bind to a protein of interest are described in the scientific literature. The method comprises the use of a library of filamentous phage containing an insertion of random nucleotide sequences of a fixed length in the gene for the pill or pVm coat protein. Transformation of bacteria with this phage library leads to expression of the phage, which displays the altered protein. A target molecule that is biotinylated, or labeled, in such a manner that it can be captured by a bead, or affixed to a surface, may then be used to capture phage displaying a coat protein that contains a specific sequence capable of binding the target molecule. See, for example, Smith, Science 228: 1315-1317 (1985); Scott and Smith, Science 249: 386-390 (1990); and Cwirla et al., Proc. Acad. Sci. U.S.A., 87: 6378-6382 (1990). The affinity of peptides that are identified as binding the NF or EV neo-epitopes using this technology can be improved by affinity maturation procedures.

Methods to Detect BACE-1 Activity

The method of the invention include the following aspects and embodiments:

Preparation of Reaction Mixture

In a first step, a biological sample or an extract prepared from a biological sample is obtained. The biological sample can be aliquots of CSF or plasma or serum obtained from an individual or an extract prepared from the CSF, plasma, or tissue, such as brain, obtained from an individual. Extracts include, but are not limited to, membrane preparations prepared from brain tissue of an individual as shown in Example 1 or BACE-1 enzyme partially or fully purified from serum or plasma, for example purified from serum using antibodies specific for the BACE-1 as shown in Example 3. The biological sample can be neat or diluted. In particular assays, serial dilutions of the biological sample or extract are made and each dilution in the series is assayed in a separate reaction.

The biological sample or extract or dilution is then contacted with a mixture comprising a labeled a peptide substrate having a BACE-1 cleavage site that has been modified to be cleaved by BACE-1 at a rate that is faster than the rate the wild-type or Swedish cleavage site is cleaved by BACE-1, for example, a BACE-1 cleavage site comprising at least the amino acid sequence NFEV (SEQ ID NO:3), and at least one aspartyl protease inhibitor that inhibits endogenous aspartyl proteases but does not inhibit BACE-1 to provide a reaction mixture. Preferably, the peptide substrate comprises the amino acid sequence EVNFEVEF (SEQ ID NO: 7). More preferably, the peptide substrate is a 10-mer that has the amino acid sequence REVNFEVEFR (SEQ ID NO: 8) or a 15-mer that has the amino acid sequence KTEEISEVNFEVEFR (SEQ ID NO: 9). It is preferable that the peptide substrate be labeled at the amino terminus.

The reaction mixture further includes a buffer to maintain the pH of the reaction at a pH of about 4.5. Optionally, the reaction mixture includes one or more additional protease inhibitors to proteases such as chymotrypsin, thermolysin, papain, pronase, pancreatic extract, and trypsin. It is preferable that the aspartyl protease inhibitor be added to the reaction mixture and the biological sample or extract before the peptide substrate is added. This reduces the likelihood that any endogenous aspartyl protease will still be active at the time the peptide substrate is added.

Incubation to Produce Peptide Product

The reaction mixture is then incubated under conditions and time sufficient for any BACE-1 activity that might be in the biological sample to cleave the peptide substrate to produce two peptide fragments: an amino terminal peptide product and a carboxy terminal peptide product. When the peptide comprises the amino acid sequence NFEV (SEQ ID NO: 3) as the BACE-1 cleavage site, BACE-1 cleaves between the F and E amino acid residues to produce two peptide fragments: an amino terminal peptide product having the amino acid sequence NF at its carboxy terminus and a carboxy terminal peptide product having the amino acid sequence EV at its amino terminus. In general, the reaction is incubated at 37° C. for between about two to four hours.

Detection of the Peptide Product

Following incubation of the reaction mixture, the labeled peptide product is detected. The labeled peptide product can be detected in a variety of ways, including immunologic, chromatographic, electrophoretic, and the like. Several methods for detecting the labeled peptide product are described below.

The labeled peptide product can be detected using a chromatographic method to separate the labeled peptide product from unreacted labeled peptide substrate and then detecting the labeled peptide product. In Example 1, the peptide substrate was labeled at the amino terminus with the fluorophore coumarin and high performance liquid chromatography (HPLC) was used to separate coumarin labeled peptide product from any uncleaved coumarin labeled peptide substrate in the reaction mixture using a solvent gradient. Detection of coumarin fluorescence of the labeled peptide product separated from the labeled peptide substrate was by a detection means for detecting fluorescence, for example, a photomultiplier detector.

The labeled peptide product can be detected using a label that is a ligand for a receptor to separate the labeled peptide product from the unreacted labeled peptide substrate and then detecting the labeled peptide product using an antibody specific for the neo-epitope that was created upon cleavage of the peptide substrate with BACE-1. As shown in Example 3, the peptide substrate is labeled at the amino terminus with biotin. After the reaction has run its course, the reaction mixture is introduced to a surface coated with streptavidin (for example, the wells of an ELISA plate or magnetic beads). The streptavidin captures the biotin-labeled amino terminal peptide product and any uncleaved biotin-labeled peptide substrate. Labeled antibody specific for neo-epitope is introduced to bind any biotin-labeled amino peptide product. After removing any unbound antibody, antibody bound to the biotin-labeled amino terminal peptide product is detected. The neo-epitope specific antibody can be labeled with a fluorphore, horseradish peroxidase, alkaline phosphatase, europium, ruthenium, radiolabel, or other label which enables detection.

Alternatively, the antibody specific for the neo-epitope is detected using another labeled antibody specific for the neo-epitope antibody. As shown in Example 2, the peptide substrate is labeled at the amino terminus with biotin. After the reaction has run its course, the reaction mixture is introduced to a surface coated with streptavidin (for example, the wells of an ELISA plate or magnetic beads). The streptavidin captures the biotin-labeled amino terminal peptide product and any uncleaved biotin-labeled peptide substrate. Labeled antibody specific for the neo-epitope is introduced to bind any biotin-labeled amino peptide product. After removing any unbound antibody, a second antibody is then introduced to detect the antibody bound to the biotin-labeled amino terminal peptide product. The second antibody can be labeled with a fluorphore, horseradish peroxidase, alkaline phosphatase, europium, ruthenium, radiolabel, or other label which enables detection.

In a further aspect, the peptide substrate is labeled at the carboxy terminus in the manner as described above and the labeled carboxy terminal peptide substrate formed upon cleavage with BACE-1 is detected as described above except that an antibody or aptamer specific for the neo-epitope created at the amino terminus of the carboxy terminal peptide product is used instead of the antibody or aptamer specific for neo-epitope created at the carboxy terminus of the amino terminal peptide product.

In preferred aspects, the peptide substrate comprises at least the amino acid sequence NFEV (SEQ ID NO:3) and antibodies specific for the NF neo-epitope is used to detect the labeled amino terminal peptide product and antibodies specific for the EV neo-epitope is used to detect the labeled carboxy terminal peptide product.

In a further aspect, the peptide substrate is labeled at the amino terminus with coumarin and at the carboxy terminus with fluorescein and the ratio of coumarin to fluorescein fluorescence is monitored during the course of the reaction. Coumarin fluoresces at 445 nm and fluorescein fluoresces at 520 nm. The ratio of fluorescence at 445 nm to fluorescence at 520 nm is determined at the beginning of the reaction and then monitored over time during the course of the reaction. An increase in the ratio over time indicates that the biological sample or extract has BACE-1 activity.

Controls

In particular aspects of the method of the present invention it is advantageous to include control reactions. For example, control reactions can include reactions that include various concentrations of a known BACE-1 inhibitor such as Merck-3 (compound 3 in Stachel et al, J. Med. Chem. 47: 6447-50 (2004)) or statine-Val (Stat(V)). These control reactions can be used to confirm that the cleavage of the peptide substrate in the reactions is a result of BACE-1 activity in the biological sample. Other control reactions include one or more reactions that include a known amount of BACE-1 or modified BACE-1, for example, BACE-1 without the transmembrane and cytoplasmic regions.

In light of the above, the novel elements of the method include but are not limited to using a peptide substrate with a BACE-1 cleavage site having high sensitivity to BACE-1 cleavage to directly measure BACE-1 enzymatic activity, for example, the peptides disclosed herein having the NFEV (SEQ ID NO:3) amino acid sequence comprising the BACE-1 cleavage site and adding to the reaction mixture and preferably to the biological fluid or extract thereof, at least one aspartyl protease inhibitor that inhibits aspartyl proteases but does not inhibit BACE-1, for example, the aspartyl protease inhibitor pepstatin A and optionally, further including in the reaction mixture at least one or more additional protease inhibitors to proteases such as chymotrypsin, thermolysin, papain, pronase, pancreatic extract, and trypsin.

Optional Elements

Other novel elements which can be included in the method include one or more of the following: (1) performing the method with aliquots of the biological sample in the presence of a BACE-1 specific inhibitor, for example, statine-val or Merck-3, to confirm that any detected protease activity in the biological fluid or tissue that produced an amino terminal peptide product with the NF neo-epitope at the carboxy terminus and a carboxy terminal peptide product with EV at the amino terminus was due to BACE-1 activity in the biological fluid and not due to activity of some other protease; (2) using a recombinant BACE-1, for example, the baculovirus-produced BACE-1, to quantify relative BACE-1 activity on a standard curve; (3) detecting BACE-1 activity in blood products (plasma, serum); and (4) using an NFEV substrate which enables direct high sensitivity measure of enzyme activity.

Preparation of Kits for BACE- 1 Activity

The present invention further provides a kit for an assay for determining whether a biological sample has β-secretase (BACE-1) activity, which comprises at least one protease inhibitor that inhibits non-BACE-1 aspartyl proteases; a peptide substrate that includes the amino acid sequence NFEV (SEQ ID NO:3) as a cleavage site for BACE-1; an antibody that is specific for an amino acid sequence NF at the carboxy terminus or the amino acid sequence EV at the amino terminus of the peptide product produced when the peptide substrate is cleaved by the BACE-1; and instructions for performing the assay. Optionally, the kit can further include a BACE-1 specific inhibitor, a recombinant BACE-1, or both.

In further aspects of the kit, the peptide substrate comprises the amino acid sequence EVNFEVEF (SEQ ID NO: 7). Further still, the peptide substrate comprises an amino acid sequence selected from the group consisting of KTEEISEVNFEVEFR (SEQ ID NO: 8) and REVNFEVEFR (SEQ ID NO: 9). In various aspects of the kit, the peptide substrate is labeled at the amino terminus, for example, the peptide substrate is labeled at the amino terminus with biotin.

In further still aspects of the kit, the protease inhibitor is pepstatin A. In further aspects, the antibody specific for the amino acid sequence NF at the carboxy terminus or EV at the amino terminus of the peptide product is labeled.

Biomarker for Alzheimer's Disease

In another embodiment of the invention, BACE-1 activity can act as a biomarker for Alzheimer's disease. The biomarker comprises measuring the 50% inhibitory concentration (IC₅₀) of a biological sample from an individual diagnosed with or suspected to have Alzheimer's disease measured against a BACE-1 inhibitor like Merck-3 and comparing this measurement to the IC₅₀ for individuals with and without Alzheimer's disease. For example, CSF or plasma or biological samples from control individuals (individuals without Alzheimer's disease or mild cognitive impairment (MCI)) and individuals with Alzheimer's disease are assayed in the presence of a BACE-1 inhibitor such as Merck-3 to obtain an intrinsic IC₅₀ for each group. The intrinsic IC₅₀ for each group provides the standard value for normal (control) verses Alzheimer's disease groups to which is compared the IC₅₀ value obtained from an individual being evaluated for Alzheimer's disease. An IC₅₀ obtained from an individual that is similar to the intrinsic IC₅₀ of that of the Alzheimer's disease group would indicate that the individual has Alzheimer's disease. Similarly, samples obtained from an individual being evaluated for Alzheimer's disease that have an IC₅₀ value that over time becomes more similar to the intrinsic IC₅₀ of an individual with Alzheimer's disease would indicate that the individual is developing Alzheimer's disease. Alternatively, the biomarker can be used to monitor the efficacy of a treatment for Alzheimer's disease. An IC₅₀ value obtained from biological samples from an individual undergoing treatment for Alzheimer's disease that when measured over time become more similar to the intrinsic IC₅₀ value of an individual without Alzheimer's disease would indicate that the progress of the disease is being abated. Thus, the present invention provides a biomarker for Alzheimer disease comprising a 50% inhibitory concentration (IC₅₀) value for a BACE-1 inhibitor obtained from a biological sample from an individual that is greater than the IC₅₀ value for a BACE-1 inhibitor from a biological sample obtained from an individual that does not have Alzheimer's disease. Endogenous BACE-1 activity may be different not only between those having Alzheimer's disease and those who are normal (no neurological disease), but may also differ from individuals with other neurological conditions, such as mild cognitive impairment (MCI). Accordingly, in another aspect the invention is a biomarker for disease progression comprising the comparison of an IC₅₀ value from an individual being tested to the intrinsic IC₅₀ value obtained for individuals with MCI. Use of the biomarker in this manner is both desirable and an objective for management of Alzheimer's disease. See, for example, the Reagan Research Institute and NIA “Consensus Report of the Working Group on: “Molecular and Biochemical Markers of Alzheimer's Disease”, Neurobiology of Aging, 19 (2): 109-116 (1998).

The present invention further provides a biomarker for Alzheimer's disease which is a truncated form of the BACE-1 enzyme. As shown in Example 4, a carboxy terminal truncated BACE-1 was identified in the CSF obtained from rhesus monkeys. The carboxy terminal truncated BACE-1 has an apparent molecular weight as determined by Tris-glycine gel electrophoresis of about 56 kDa. The carboxy terminal truncated BACE-1 is believed to comprise at least the first 460 amino acid residues of the BACE-1 because antibodies specific for epitopes within the first 460 amino acids can capture the truncated BACE-1 from rhesus monkey CSF whereas antibodies specific for epitopes following amino acid residue 460 cannot capture the truncated BACE-1 from rhesus monkey CSF.

The carboxy terminal truncated BACE-1 is believed to be useful as a biomarker for determining whether an individual has Alzheimer's disease or is at risk of developing Alzheimer's disease, for determining the efficacy of a therapeutic procedure being used to treat an individual who has Alzheimer's disease, or for evaluating the efficacy of a drug candidate for treating Alzheimer's disease, or other biomarker use as described in the Reagan Research Institute and NIA “Consensus Report of the Working Group on: “Molecular and Biochemical Markers of Alzheimer's Disease”, Neurobiology of Aging, v19 (2): 109-116 (1998).

Therefore, the present invention further provides a method for detecting Alzheimer's disease in an individual or an individual at risk for developing Alzheimer's disease comprises obtaining a biological fluid from the individual; and, detecting the carboxy terminal truncated BACE-1 having an apparent molecular weight of about 56 kDa in the biological fluid, wherein the presence of the carboxy terminal truncated BACE-1 indicates the individual has Alzheimer's disease.

The present invention further provides a method for determining the efficacy of a treatment for Alzheimer's disease in an individual comprises obtaining a biological fluid sample from the individual before treatment and at various times during the treatment; and detecting a carboxy terminal truncated BACE-1 having an apparent molecular weight of about 56 kDa in the biological fluid samples; and, comparing the amount of the carboxy terminal truncated BACE-1 in samples obtained from the individual at various times during the treatment to the amount in the sample before the treatment to determine the efficacy of the treatment.

Further still, the present invention provides a method for determining the efficacy of a drug candidate for treating Alzheimer's disease comprises obtaining biological fluid samples from the individual before treatment with the drug candidate and at various times during treatment with the drug candidate; and detecting a carboxy terminal truncated BACE-1 having an apparent molecular weight of about 56 kDa in the biological fluid samples; and, comparing the amount of the carboxy terminal truncated BACE-1 in samples obtained from the individual at various times during the treatment with the drug candidate to the amount in the sample before the treatment with the drug candidate to determine the efficacy of the drug candidate for treating Alzheimer's disease.

The above methods for detecting the carboxy truncated BACE-1 can be performed in conjunction with the methods disclosed herein for detecting BACE-1 activity or as a component of a multi-analyte panel comprising other markers for Alzheimer's disease, including for example, but not limited to, Aβx-42 and sAPPα.

The following examples are intended to promote a further understanding of the present invention.

### EXAMPLE 1

This example illustrates an assay for detection of BACE-1 activity in the brain.

Brain BACE-1 enzymatic activity was measured using brain membrane extracts in a two-step method. In the first reaction step, a brain membrane extract containing endogenous BACE-1 is reacted with a coumarin labeled 10-mer peptide substrate in the presence of non-specific protease inhibitor cocktail and aspartyl protease inhibitor Pepstatin A. In a second detection step, an HPLC-based detection method is used to determine the extent of enzymatic cleavage of the 10-mer substrate.

Brain membranes are prepared as follows. Mice brain sections are homogenized in 10× w/v 50 mM MES buffer with protease inhibitor (Roche Applied Science, Indianapolis, Ind., Cat#11836145), pH 6.0 using a glass/Teflon homogenizer, using about 10 strokes. The homogenates are then spun at 2000 rpm (900×g) at 4° C. for 10 minutes. The supernatant is discarded and the pellet is further homogenized with 2× v/v volume 50 mM MES buffer as above using a glass/Teflon homogenizer, again about 10 strokes. The homogenate is transferred to ultracentrifuge tubes and spun at 35K rpm (100K g) at 4 C for 1 hour using a TLA-55 rotor (Beckman Instruments, Fullerton, Calif.). The supernatant is discarded and the pellet is resuspended in 4 ml of MES buffer. Protein concentration is determined using modified Bradford method (DC protein assay kit Cat#500-0114, Bio-Rad Laboratories, Hercules, Calif.).

The amount of BACE-1 in the membrane preparation is determined by semi-quantitative Western blotting using Baculovirus expressed extracellular region of BACE-1 consisting of amino acids 1 to 460 (baculo-BACE) as a standard (See Shi et al., J. Biol. Chem. 276: 10366-10373 (2001); Shi et al., J. Biol. Chem. 278: 21286-21294 (2003)). Fifty μg of protein is loaded onto 10% Tris-glycine polyacrylamide gel and run at 125V for 90 minutes. The protein is transferred onto 0.45 μm PVDF membrane at 12V at 4° C. overnight. The membrane is then blocked with ODYSSEY blocking buffer (Li-Cor Biosciences, Lincoln, Nebr., Cat. #927-40000) at room temperature for one hour. The membrane is then incubated with anti-BACE-1 antibody (anti-BACE-1 antibody JHU070604 can be obtained from Phil Wong, Johns Hopkins University, Baltimore, Md.) at a 1:1000 dilution in ODYSSEY buffer with 0.1% Tween-20 for two hours at room temperature. After thorough washing (PBS with 0.05% Tween-20), the BACE-1 protein is detected using Alexa-680 goat anti rabbit antibody (1:2500 dilution in ODYSSEY buffer with 0.1% Tween-20) for one hour at room temperature. Following the above protocol produced the results shown below.

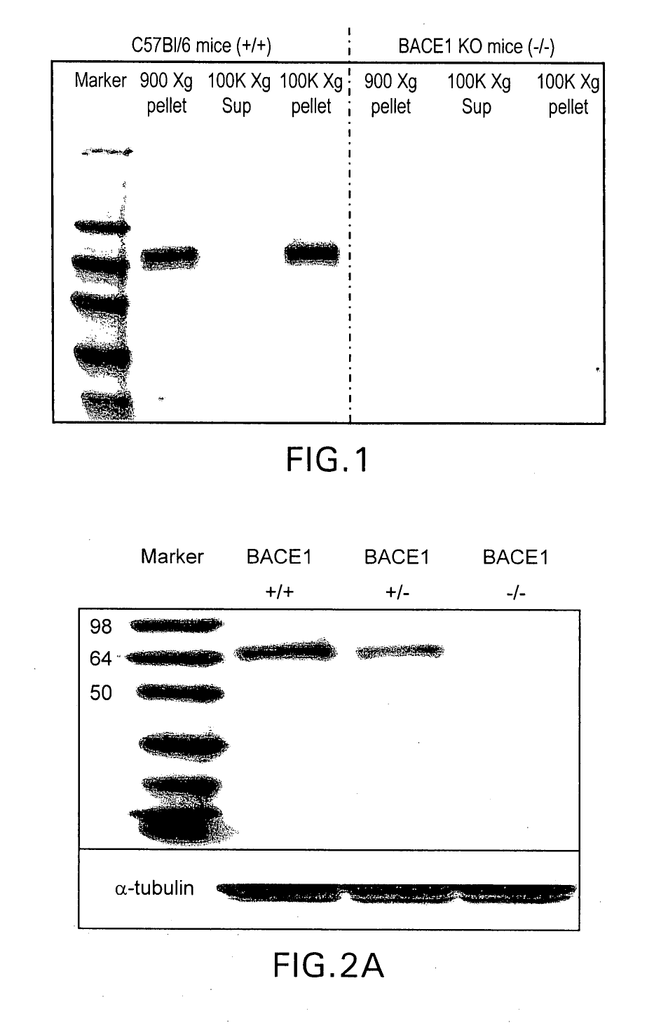

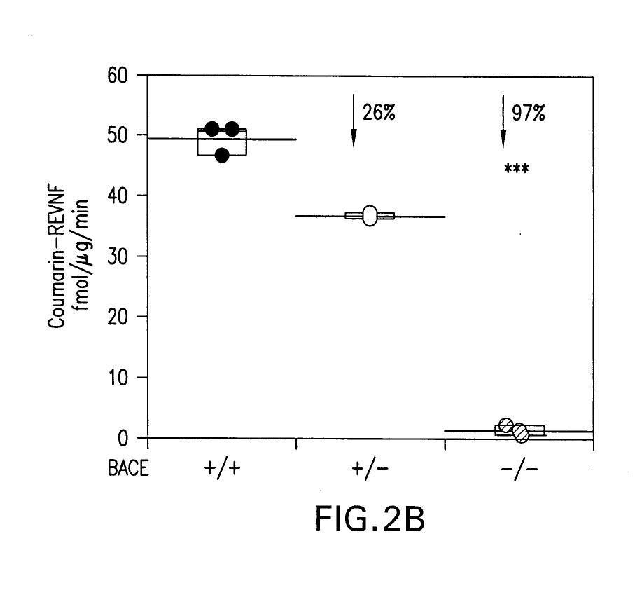

FIG. 1 shows a Western blot of MES buffer membrane preparations prepared from wild-type (WT) and BACE-1 knockout (KO) mice and probed with the antibody JHU070604. The Western blot shows that there is no detectable BACE-1 in membranes prepared from the BACE-1 KO mice. FIG. 2A shows a Western blot showing the relative expression levels of BACE-1 in WT, heterozygote, and homozygote BACE-1 KO mice. The Western blot shows that the amount of BACE-1 expression is proportional to the number of BACE-1 alleles in the mouse. FIG. 2B shows that in an ex-vivo BACE enzymatic activity assay, as described in this example, that homozygous BACE-1 KO mice have no measurable BACE-1 activity and that heterozygous mice have about 26% less of the BACE-1 activity compared to the BACE-1 activity in the wild-type mice (See, Cai et al., Nature Neurosci. 4: 233-234 (2001).

In the reaction first step, the BACE-1 enzymatic reaction was performed using a coumarin-labeled 10-mer peptide substrate having the amino acid sequence REVNFEVEFR (SEQ ID NO:8). BACE-1 isolated from brain homogenates is set up in a reaction to assess enzymatic activity. About 200 μg of total extracted protein from brain homogenate is reacted with 1 μM of the coumarin-labeled peptide substrate in reaction buffer containing 50 mM NaOAc, 0.01% BSA, 15 mM EDTA, 0.2% CHAPS (Pierce Chemical Co., Rockford. Ill., Cat#28300), 1 mM Deferoxamine Mesylate (Sigma-Aldrich, Inc., St. Louis, Mo., Cat# D9533), and 10 μM pepstatin A (Calbiochem, San Diego, Calif., Cat #516481) at pH 4.5 for 37° C. for three hours. Optionally, about 10 μL of 10× Protease Inhibitor (1× final) (Roche Diagnostics GmbH, Cat#11836153001; provides 1.5 μg/mL chymotrypsin, 0.8 μg/mL thermolysin, 1 mg/mL papain, 1.5 μg/mL pronase, 1.5 μg/mL pancreatic extract, and 2 ng/mL trypsin) can be added. In parallel, when desired control reactions are set up using baculovirus-expressed BACE-1 at a concentration of 200 pM. If the sample contains BACE-1 activity, then the coumarin-labeled 10-mer is cleaved between the F and E amino acid residues producing a coumarin-labeled 5-mer peptide product ending with the amino acid sequence NF and an unlabeled 5-mer peptide beginning with the amino acid sequence EV.

In the second step, the BACE-1 cleavage product is detected. The resulting mixture is then chromatographed in a Waters Alliance HT HPLC system (Waters 2790, Waters Corporation, Milford, Mass.) to detect product and substrate fluorescence as follows. The samples are placed in a 96 deep-well plate. Then, 25 μL of samples is then injected into a ZORBAX extend C18 column (2.1×150 mm) (Cat#186000434, XTERRA, Waters Corporation). The substrate and the cleaved product are separated using a gradient created with the following 2 solvents: solvent A was 0.045% TFA (Cat# AX0142-1, EMD Biosciences, San Diego, Calif.) in water and solvent B was 0.045% TFA in acetonitrile (Cat#269778, Sigma-Aldrich Inc.). The ratio of solvent A to B is programmed to be 80% to 20% in the first three minutes, 70% to 30% in the next four to five minutes, 5% to 95% in the next six to seven minutes and 80% to 20% in the final eighth minute.

In general, the BACE-1 cleavage product of the 10-mer peptide substrate, Coumarin-labeled REVNF peptide product (SEQ ID NO:10), is observed in the four to five minute gradient of the run while the uncleaved substrate was observed in the six to seven minute gradient of the run. The amount of cleaved product and substrate are detected with excitation at 340 nm and emission at 440 nm. On average, hydrolysis was observed to be about 15% of total substrate that had been added in the reaction.

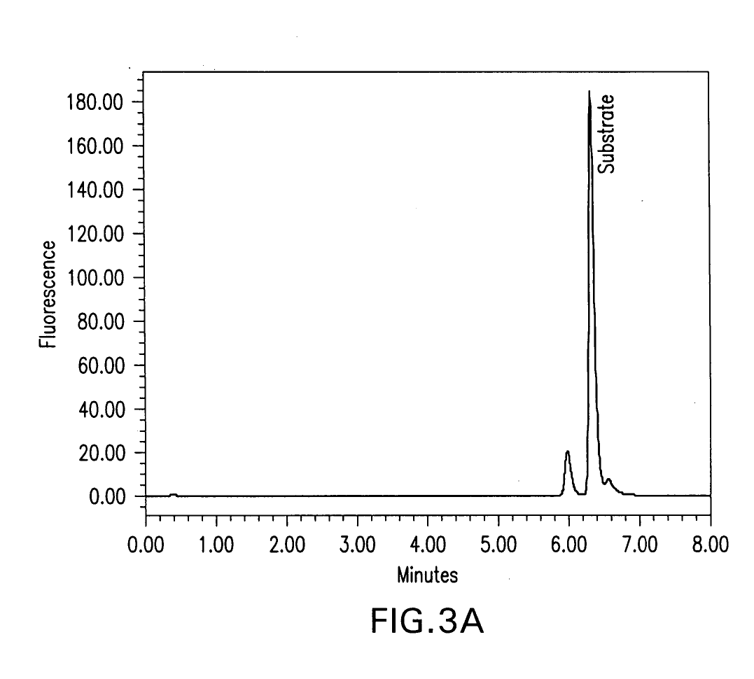

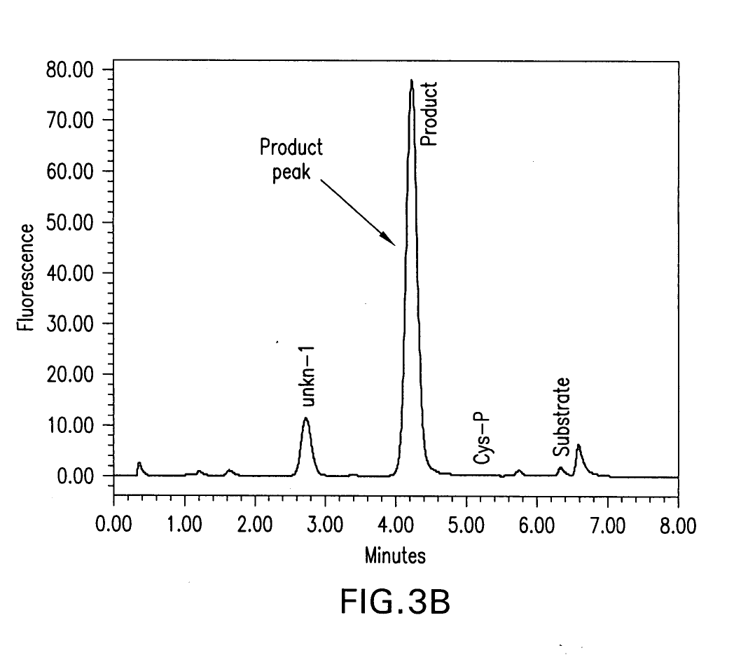

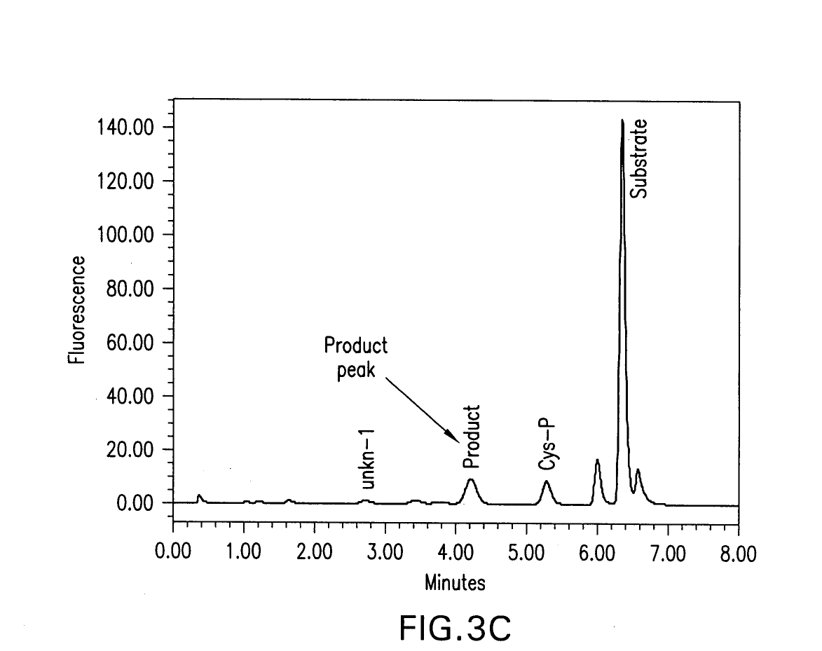

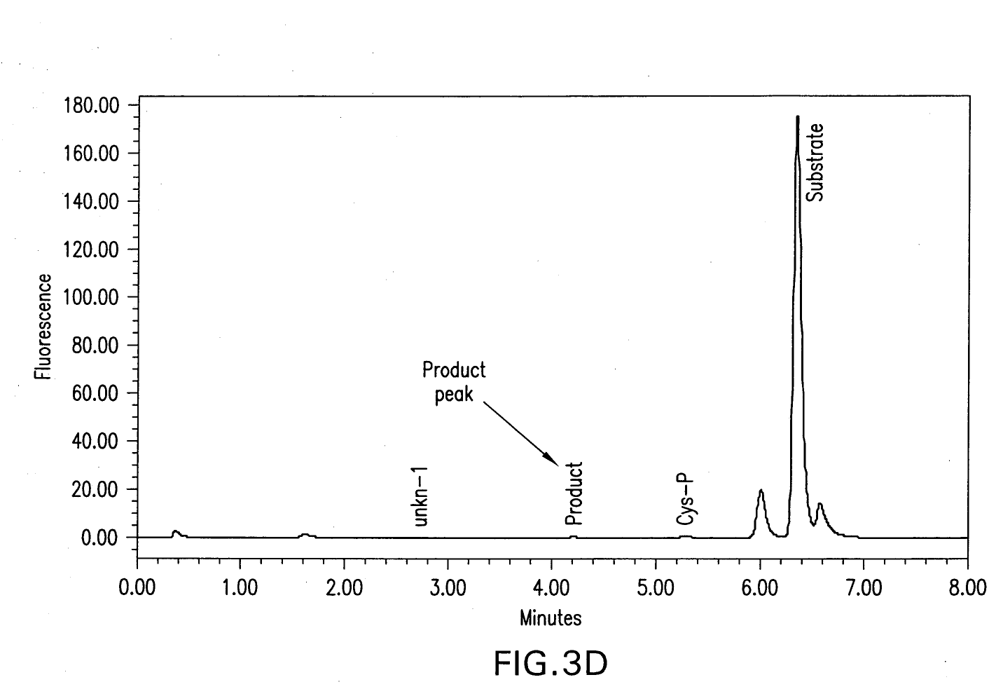

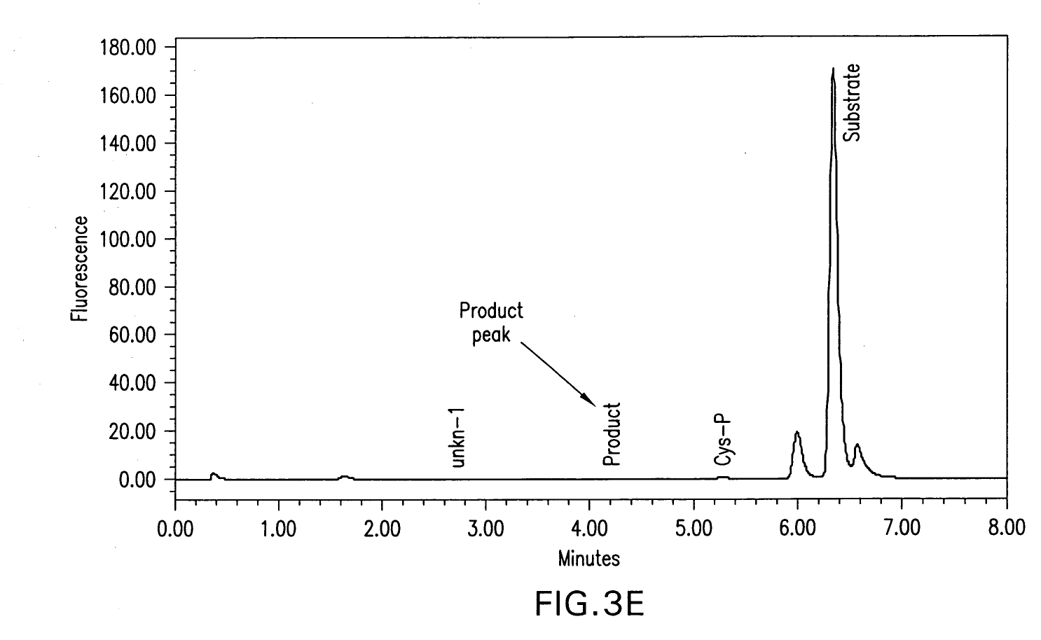

FIGS. 3A to 3E show typical HPLC traces showing the peaks observed in the presence of Coumarin-labeled 10-mer substrate only (FIG. 3A), brain extract with substrate and protease inhibitors (FIG. 3B); brain extract with substrate, protease inhibitors, and 10 μM pepstatin A (FIG. 3C); brain extract with substrate, protease inhibitors, 10 μM pepstatin A, and 10 μM statine-Val (Stat(V), a peptide BACE-1 inhibitor) (FIG. 3D); and, brain extract with substrate, protease inhibitors, 10 μM pepstatin A, and 10 μM BACE-1 inhibitor Merck-3 (FIG. 3E). BACE-1 specific activity was measured in the presence of 10 μM Pepstatin A. FIGS. 3D and 3E, BACE-1 inhibitor's Stat(V) and Merck-3 led to complete abolition of substrate cleavage. The Figures show that cleavage of the substrate is solely due to BACE-1 activity in the brain membrane preparations. Thus, the assay can readily detect BACE-1 activity in brain membrane preparations.

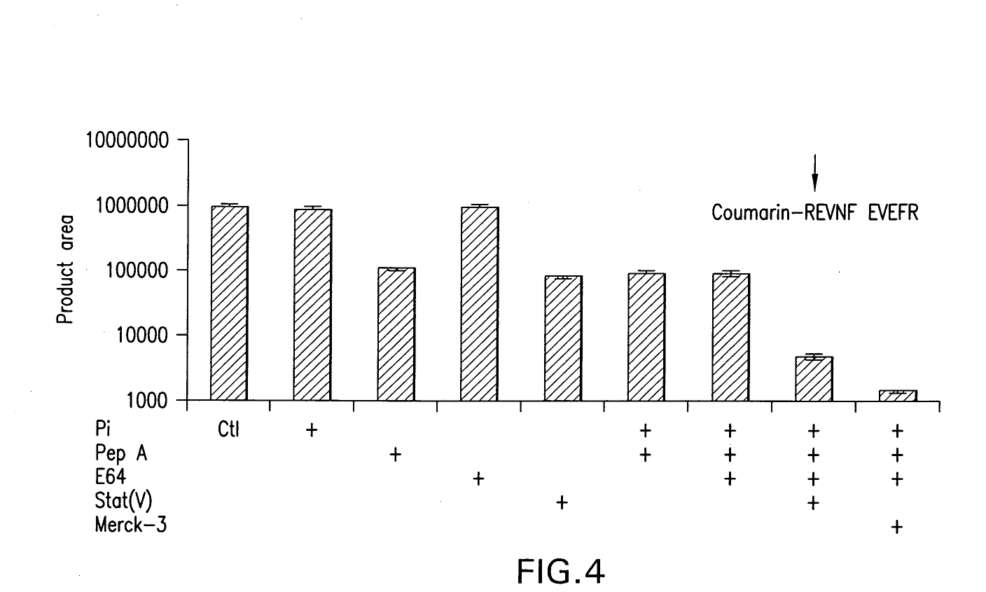

FIG. 4 shows brain BACE-1 activity measured as the area of product peak for membrane preparations from WT mice. Non-specific aspartyl protease activity was abolished by Pepstatin A while BACE-1-specific activity was abolished by Stat(V) and completely by Merck 3 at 10 μM concentration. Cysteine protease inhibitor E64 at 10 μM has no effect on cleavage of substrate.

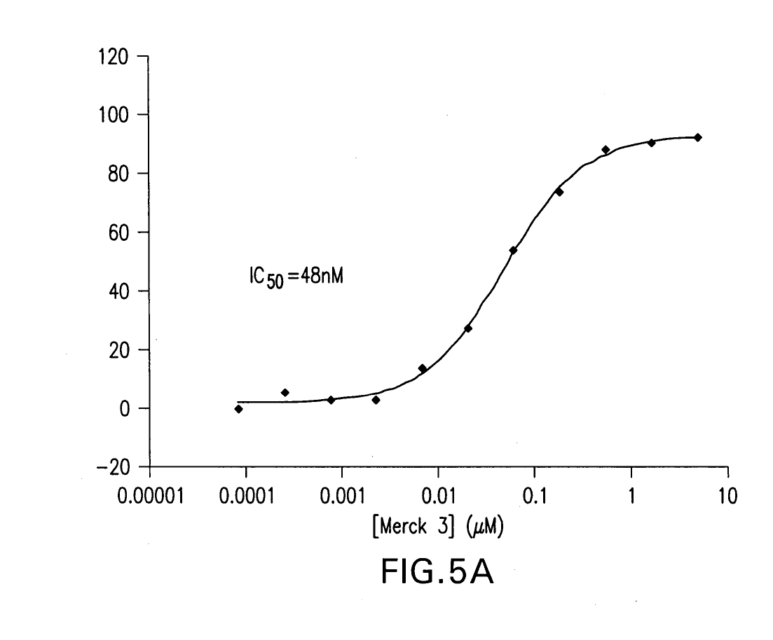

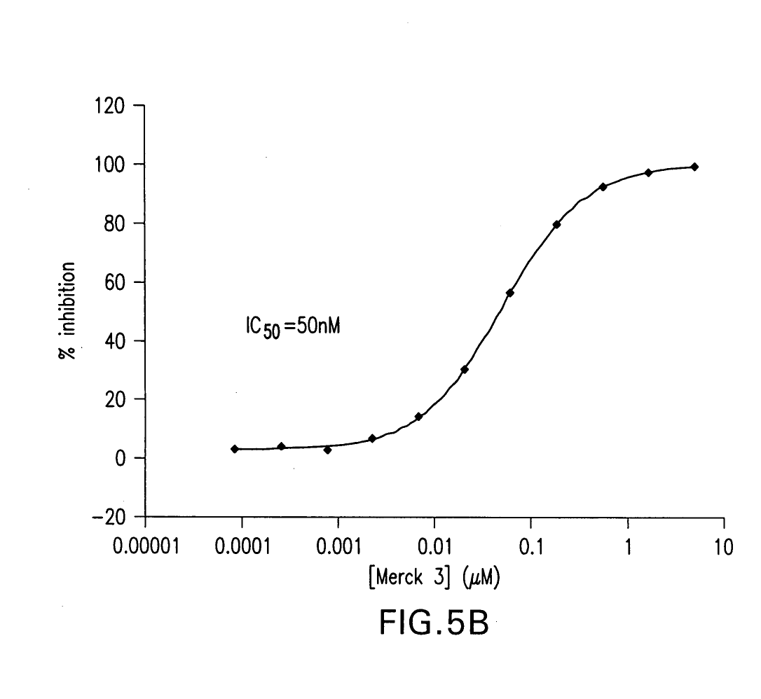

FIG. 5A shows a dose-dependent titration of brain BACE-1 activity in the presence of the BACE-1 specific inhibitor Merck-3 using brain membrane preparations from WT mice. Shown in FIG. 5B for comparison is a dose-dependent titration of BACE-1 activity in the presence of the Merck-3 using baculovirus-expressed BACE-1.

This example shows that the assay can detect BACE-1 activity in membrane preparations extracted from brain tissue.

### EXAMPLE 2

This example illustrates a method for solution based detection of BACE-1 activity in cerebral spinal fluid (CSF). BACE-1 enzymatic activity in CSF was measured using a two-step method. In the first step, cleavage of a biotinylated peptide substrate is accomplished using CSF as the source of BACE-1 enzyme. In the second step, the extent of enzymatic cleavage of substrate is detected using an avidin-biotin complex and enzyme linked immunosorbent assay (ELISA).

In the peptide substrate cleavage step, 25 μL of either purified recombinant baculovirus expressed BACE-1(aa1-460) at a range of concentration from 0.8 pM to 100 pM or rhesus or human CSF is added to the wells of a 96 well assay plate (COSTAR brand, Cat#3365). To each of these wells, 25 μL of reaction buffer containing 50 mM NaOAc, 0.01% BSA, 15 mM EDTA, 0.2% CHAPS (Pierce Chemical Co., Rockford, Ill., Cat#28300), ImM Deferoxamine Mesylate (Sigma-Aldrich, Inc., Cat# D9533) and 10 μM pepstatin A (Calbiochem, San Diego, Calif., Cat #516481) at pH 4.5 is added. Optionally, about 10 μL of 10× Protease Inhibitor (1× final) (Roche Diagnostics GmbH, Cat#11836153001; provides 1.5 μg/mL chymotrypsin, 0.8 μg/mL thermolysin, 1 mg/mL papain, 1.5 μg/mL pronase, 1.5 μg/mL pancreatic extract, and 2 ng/mL trypsin) can be added. The plate is gently agitated on a shaker for 15 minutes in order to block any non-BACE-1 aspartyl protease activity by pepstatin A. Finally, 100 μL of 200 nM biotin-labeled substrate (N-terminal biotin-labeled peptide KTEEISEVNFEVEFR (SEQ ID NO:9)) prepared in reaction buffer with 10 μM pepstatin A is added. The plate is sealed tightly and incubated at 37° C. and agitated at 40 rpm for 2.5 hours. The enzymatic reaction is then arrested by adding 50 μL of 1M Tris (pH 8.0). If the sample contains BACE-1 activity, then the biotin-labeled 15-mer is cleaved between the F and E amino acid residues producing a biotin-labeled 10-mer peptide product ending with the amino acid sequence NF and an unlabeled 5-mer peptide beginning with the amino acid sequence EV.

In the second step, the product of BACE-1 enzymatic cleavage of the biotin-labeled peptide is measured by ELISA. The above reaction mixture is transferred to a streptavidin coated black plate (High binding capacity, Pierce Chemical Co., Cat#15503) and incubated overnight at 4° C. The following day, the plate is washed three times with phosphate buffered saline (PBS) with 0.1% Tween-20 (PBST) at pH 7.4. This is followed by addition of 100 μL of neo-epitope anti-NF antibody (NF carboxy-terminal neo-epitope specific rabbit polyclonal IgG that was prepared using the antigenic peptide CSEVNF (SEQ ID NO:12) as the antigen) at 1:30,000 dilution in 0.1% Tween-20 in SUPERBLOCK PBS (Pierce Chemical Co., Cat#37515) to detect the BACE-1 cleavage product of the 15-mer peptide substrate, KTEEISEVNF (SEQ ID NO:11), and incubated for 1 hour at room temperature. All of the biotinylated peptides bind the streptavidin-coated black plate, both the biotin-labeled 15-mer and the biotin-labeled peptide product. However, only the biotin-labeled 10-mer peptide can bind the anti-NF antibody.

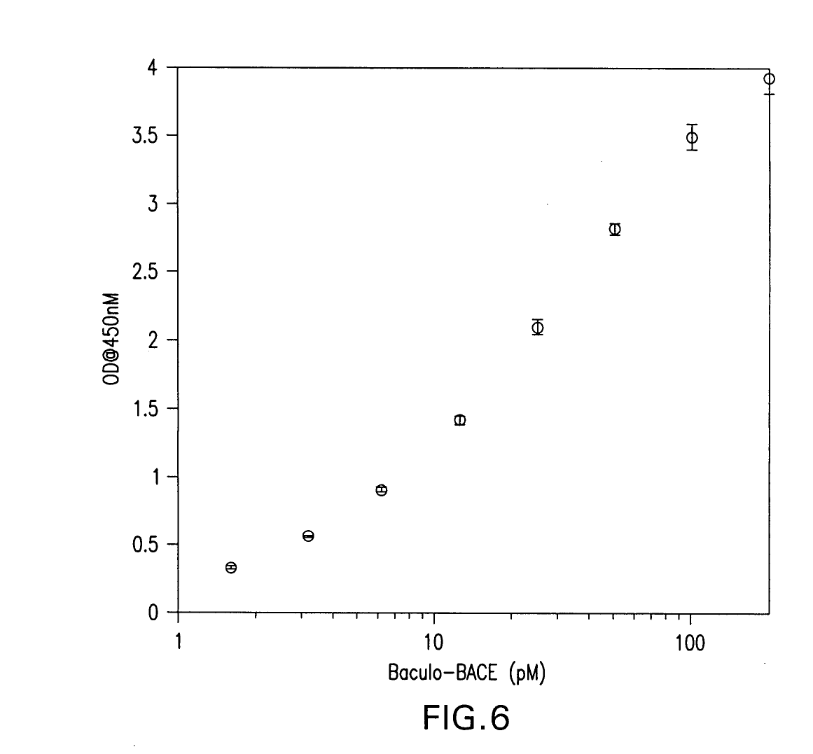

Afterwards, the plate is washed three times with PBST. Then, to detect any anti-NF antibody bound to the 10-mer peptide, 100 μL of goat anti-rabbit IgG-HRP or anti-rabbit IgG-AP (Bio-Rad, cat#170-6518) at 1:30,000 dilution in 0.1% Tween 20-SUPERBLOCK is added and incubated for one hour at room temperature. The plate is then washed five times with PBST. The reaction is finally developed using 100 μL/well of CDP-STAR ready-to-use with HRP substrate (EMD Biosciences, Inc. San Diego, Calif.) or SAPPHIRE-II Enhancer substrate (Applied Biosystems, Foster City, Calif., Cat# T2214) for 30 minutes at room temperature. Absorbance or Luminescence counts, respectively, are measured in LJL-Analyst (Molecular Devices Corp., Sunnyvale, Calif.). The counts from individual CSF samples are converted to BACE-1 concentration using coefficients determined by a quadratic fit to the baculo-BACE-1 standard curve (FIG. 6). The baculo-BACE-1 standard curve showed good titration of BACE-1 enzymatic activity with the amount of baculo-BACE-1 concentration. Baculo-BACE-1 standard curves were used in all studies to get a relative concentration of BACE-1 in human CSF samples.

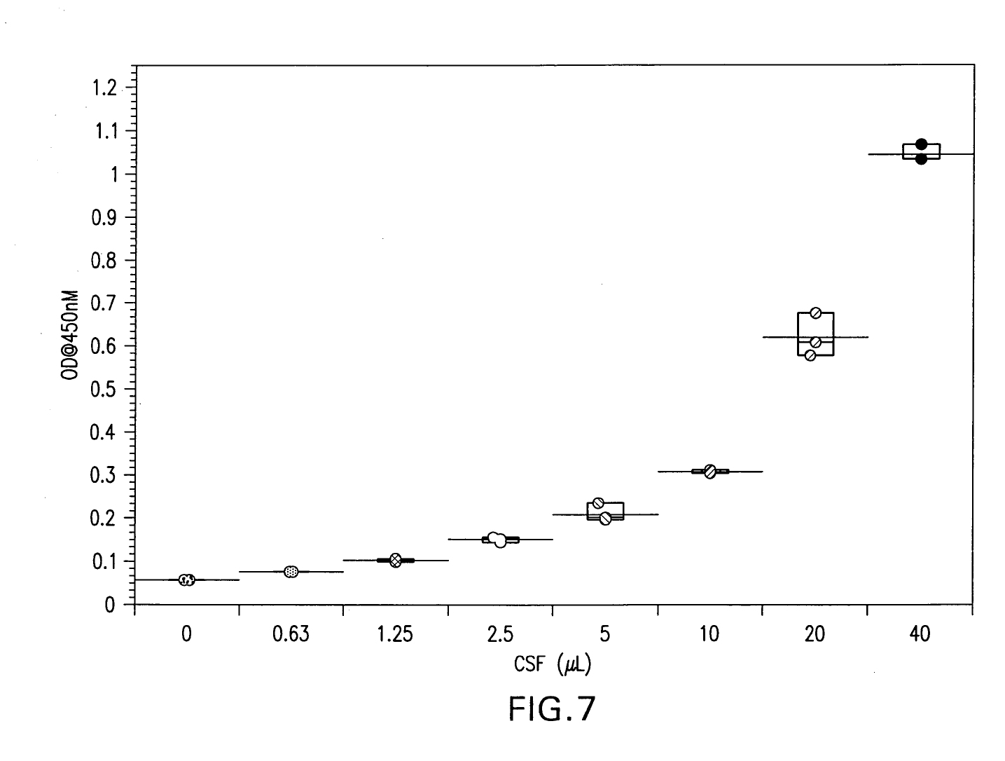

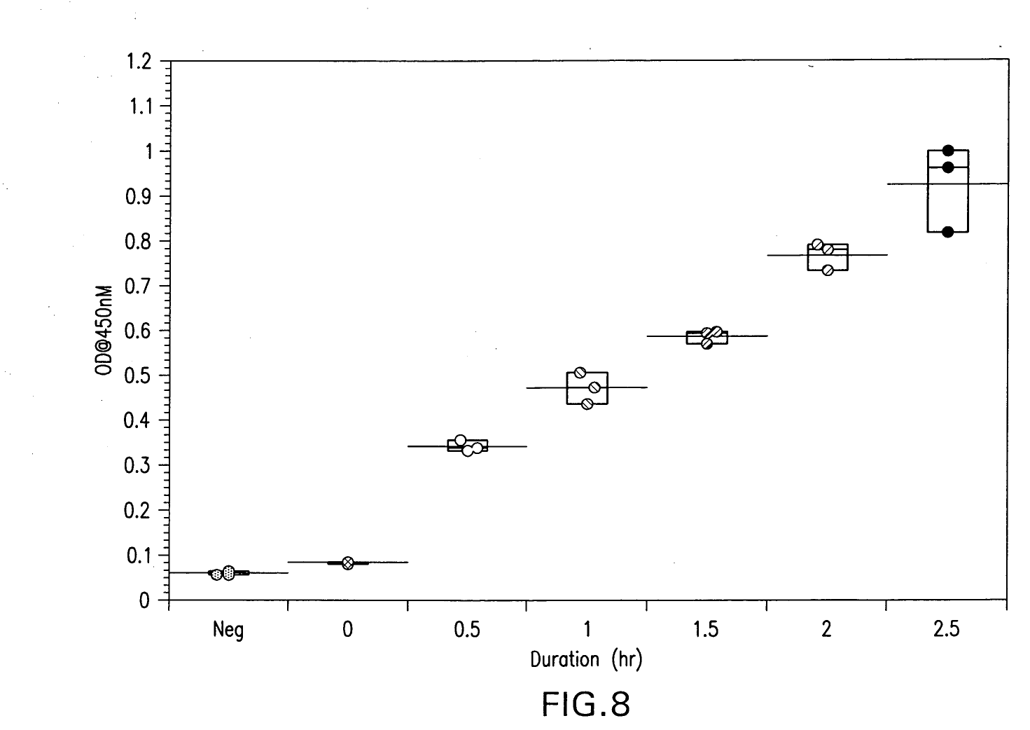

Using the above protocol, detection of BACE-1 activity in CSF was found to be a function of the volume of CSF used in the assay and the duration of the assay. Titration of CSF BACE-1 activity as a function of amount of CSF volume is shown in FIG. 7 and titration of CSF BACE-1 activity as a function of incubation time is shown in FIG. 8. Based on the results shown, using 25 μL of CSF volume and a reaction time of 2.5 hours was determined to provide good results.

To determine whether cleavage of the peptide substrate was due to BACE-1 activity in CSF and not to non-specific aspartyl proteases or other proteases, BACE-1 activity was measured in the presence of various combinations of protease inhibitors (PI), non-specific aspartyl protease activity Pepstatin A (10 μM), and the BACE-1-specific inhibitor Merck-3 (10 μM). The results, which are shown in FIG. 9, show that non-specific aspartyl protease activity accounted for about 59% of the protease activity in CSF and that the non-specific aspartyl protease activity could be abolished by adding Pepstatin A to the reaction. The remaining activity was shown to be BACE-1 activity because it could be completely abolished with BACE-1-specific inhibitor Merck-3 at 10 μM. The abolition of BACE-1 activity with Merck-3 was equivalent to that observed by heat-inactivation of the sample. The results further show that the BACE-1-specific activity in CSF occurs within a 20-fold window. Thus, the method provides a highly sensitive and specific assay for detecting BACE-1 activity.

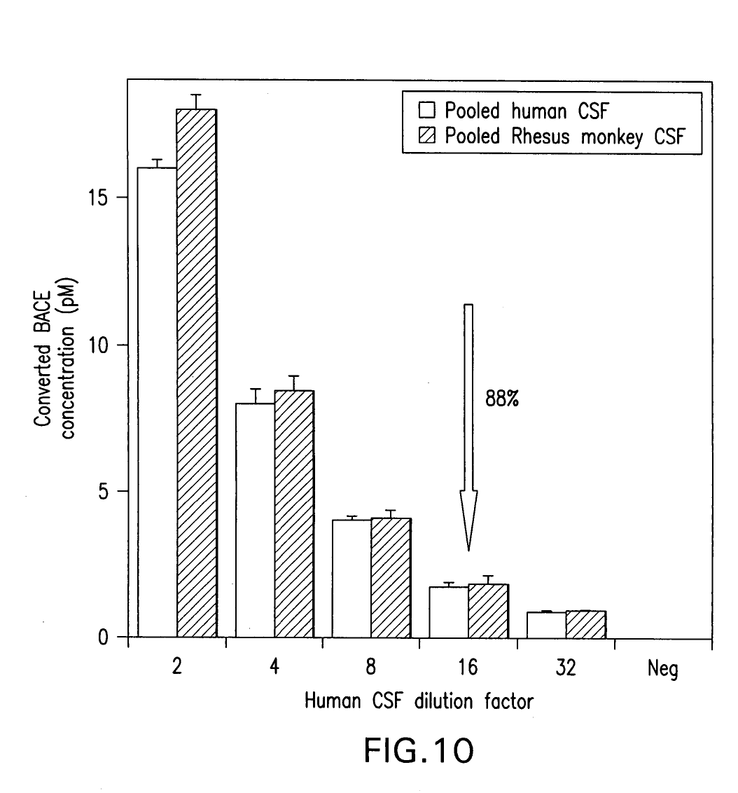

FIG. 10 shows that serial dilution of human and rhesus CSF samples diluted 1:2 can be further diluted at least eight-fold with reliable CV (<15%) and can detect a greater than 80% signal reduction. The s/n of this assay was about 70 for human CSF samples.

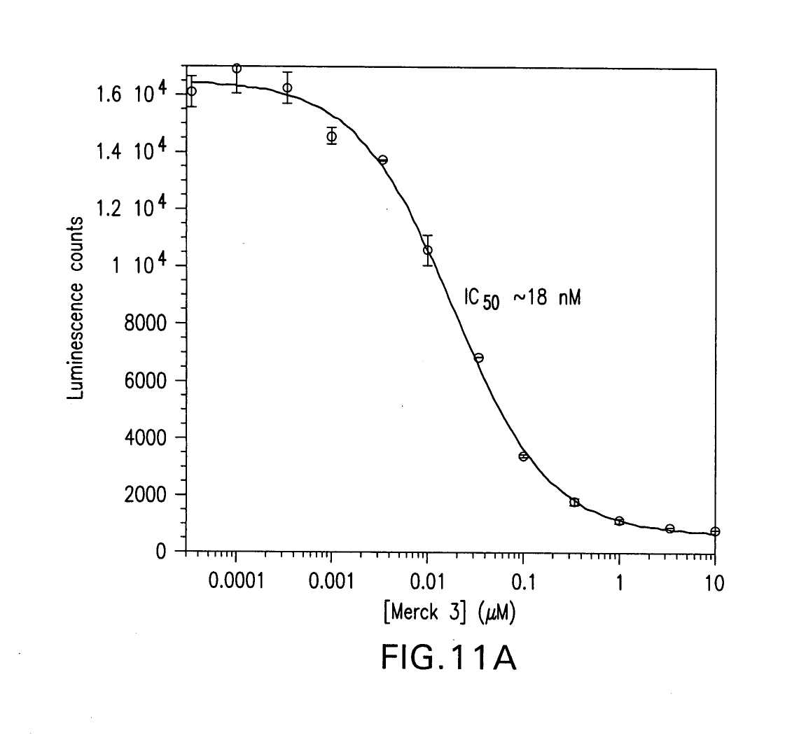

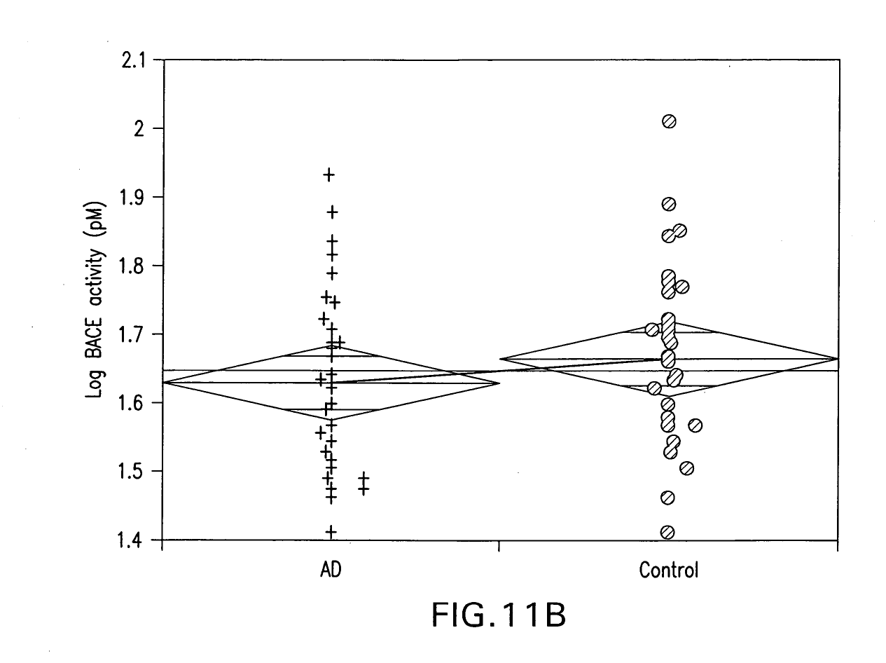

Dose-dependent inhibition of human CSF BACE-1 activity is shown in FIG. 11A. The IC₅₀ for inhibition of CSF BACE-1 activity was about 18 nM with Merck-3. In comparison, in vitro Baculo-BACE-1 enzymatic activity was inhibited with an IC₅₀ of about 11 nM while activity measured in cells was inhibited with an IC₅₀ of about 30 nM.

FIG. 11B demonstrates the application of the present invention to actual clinical human Alzheimer's disease and control CSF. Age-matched groups of approximately 29 CSF samples from autopsy confirmed Alzheimer's disease subjects were acquired and assayed for CSF BACE-1 activity. The BACE-1 activity was compared to an age-matched control cohort. Sample enzyme activity was passed through a recombinant standard curve like that shown in FIG. 6. However, unlike in Holsinger et al., Annals Neurol. 55: 898-899 (2004) or Verheijen et al., Clin. Chem. 52: E-published Apr. 13, 2006), a meaningful increase in CSF BACE-1 activity in Alzheimer's disease subjects in this data set was not observed, rather a meaningful decrease was observed when age was adjusted as a co-variant (p<0.03). It should be noted these samples were from post-mortem confirmed Alzheimer subjects with larger samples size then either of the cited other studies.

This example shows that BACE-1 activity can be detected, quantitated against a recombinant standard curve, and monitored in CSF using the above assay.

### EXAMPLE 3

This example illustrates a method for solution based detection of BACE-1 activity in human blood serum or plasma. BACE-1 enzymatic activity in the human blood serum or plasma was measured in a three step process. The first step involves an immuno-capture step using Protein-A plates and anti-BACE-1 antibody in order to isolate BACE-1 enzyme. The second step involves a cleavage reaction of biotinylated BACE-1 peptide substrate by the immuno-captured enzyme. The third step involves detection of the cleavage product using a specific detection antibody.

First Step is the BACE-1 Immuno-Capture from Plasma.

One hundred μL of 8 μg/mL anti-BACE-1 antibody EE-17 (Sigma-Aldrich, Inc. Cat# B0681) is diluted in Binding Buffer (50 mM HEPES, 150 mM NaCl, 0.1% BSA, 0.1% Tween-20, pH 7.5) and added to each well of a REACTI-BIND Protein A coated plate (Pierce Chemical Co., Cat#15130). The plates are covered with plate sealers and then incubated at 4° C. over night with gentle shaking/rotating. After the incubation, the primary IgG is removed from the plate and 100 μL of 10 μg/mL rabbit IgG (Sigma-Aldrich, Inc., Cat# I-5006) diluted in Binding Buffer, pH 7.5 is added to each well of the plate to block all protein A sites. The plates are then incubated at room temperature for about 1.5 hour with shaking. The plates are then washed with 200 μL/well of PBS containing 0.05% Tween-20 three times and then washed with 200 μL/well of PBS two times. After washing, 300 μL of human serum or plasma (Bioreclamations Inc.) are added in each well in order to capture BACE-1 from the serum or plasma onto the plates. Plates are covered with plate sealers and incubated at 4° C. over night with gentle shaking/rotating. After the incubation, the plates are washed with 200 μL/well PBS containing 0.05% Tween-20 two times, then washed with 200 μL per well PBS two times, and finally with 200 μL per well distilled H₂O two times.

Second Step is the Cleavage Reaction.

The cleavage reaction is set up as follows. Each 100 μL/well reaction mixture contains 25 μL of 0.2 M Ammonium Acetate buffer, pH 4.5 (50 nM final); 10 μL of 10× Protease Inhibitor (1× final) (Roche Diagnostics GmbH, Cat#11836153001; provides 1.5 μg/mL chymotrypsin, 0.8 μg/mL thermolysin, 1 mg/mL papain, 1.5 μg/mL pronase, 1.5 μg/mL pancreatic extract, and 2 ng/mL trypsin); 10 μL of 1 mg/mL BSA (0.1 mg/mL final); 2 μL of 10% CHAPS (0.2% final) (Sigma-Aldrich, Inc., Cat#C-5070); 1 μL of 1 mM Pepstatin A (10 μM final) (Sigma-Aldrich, Inc., Cat# P-5318); 1 μL of DMSO or 1 μL of 1 mM Merck-3 (10 μM final, negative control); 1 μL of 25 μM N-terminus-labeled biotin-labeled 15-mer peptide substrate KTEEISEVNFEVEFR (SEQ ID NO:9) (250 nM final); and 50 μL distilled H₂O. After setting up the reactions, the plates are covered with plate sealers and incubated at 37° C. over night with gentle shaking/rotating. Then the reaction mix is transferred to a 96 well round-bottom polypropylene plate (Costar 3365).

Third Step is Detection.

Detection in this example was by electrochemiluminescence (ECL) using ruthenylated neo-epitope anti-NF antibody; however, other detection methods can be used (for example, the detection method of Example 2 can used). To set up a detection reaction for ECL, ruthenylated neo-epitope anti-NF antibody is diluted to the appropriate concentration (which varies with batch of IgG and conjugate prepared) in BSA diluent (PBS with 0.1% Tween-20, 0.1% BSA, 0.05% Azide at pH 7.8). Neo-epitope anti-NF antibody can be labeled ruthenium via its primary amine group to the ruthenium (Ori-Tag-NHS ester, Cat#110034, Igen Inc., Gaithersburg, Md.) to form a stable amide group at pH about 8.5. The labeled antibody is purified from unconjugated ruthenium using size-exclusion column (PD-10, GEHealthcare). U.S. Pat. No. 5,958,783 describes metal complexes with a charged linker and their use as luminescent marker groups in immunoassays. Then, streptavidin-coated DYNABEADS brand beads (BioVeris Corporation, Gaithersburg, Md., Cat#110029) are added to achieve concentration of 200 μg/mL from 10 mg/mL bead stock. After mixing the anti-NF antibody and beads, 25 μL of the mixture is added per 100 μL reaction in each well. The plates are covered with aluminum foil and incubated with shaking at room temperature for about 3 hours. If the sample contains BACE-1 activity, then the biotin-labeled 15-mer is cleaved between the F and E amino acid residues producing a biotin-labeled 10-mer peptide product ending with the amino acid sequence NF and an unlabeled 5-mer peptide beginning with the amino acid sequence EV. All of the biotinylated peptides bind the streptavidin-coated beads, both the biotin-labeled 15-mer and the biotin-labeled peptide product. However, only the biotin-labeled 10-mer peptide product can bind the ruthenium-labeled anti-NF antibody.

Following the incubation, 125 μL of PBS buffer, pH 7.4 per well are added and samples are analyzed using an Origen Igen M384 machine (IGEN International, Gaithersburg, Md.) in which the beads are magnetically separated from unbound material and ECL associated with any the ruthenium-labeled antibody bound to the 10-mer peptide product bound to the beads is measured.

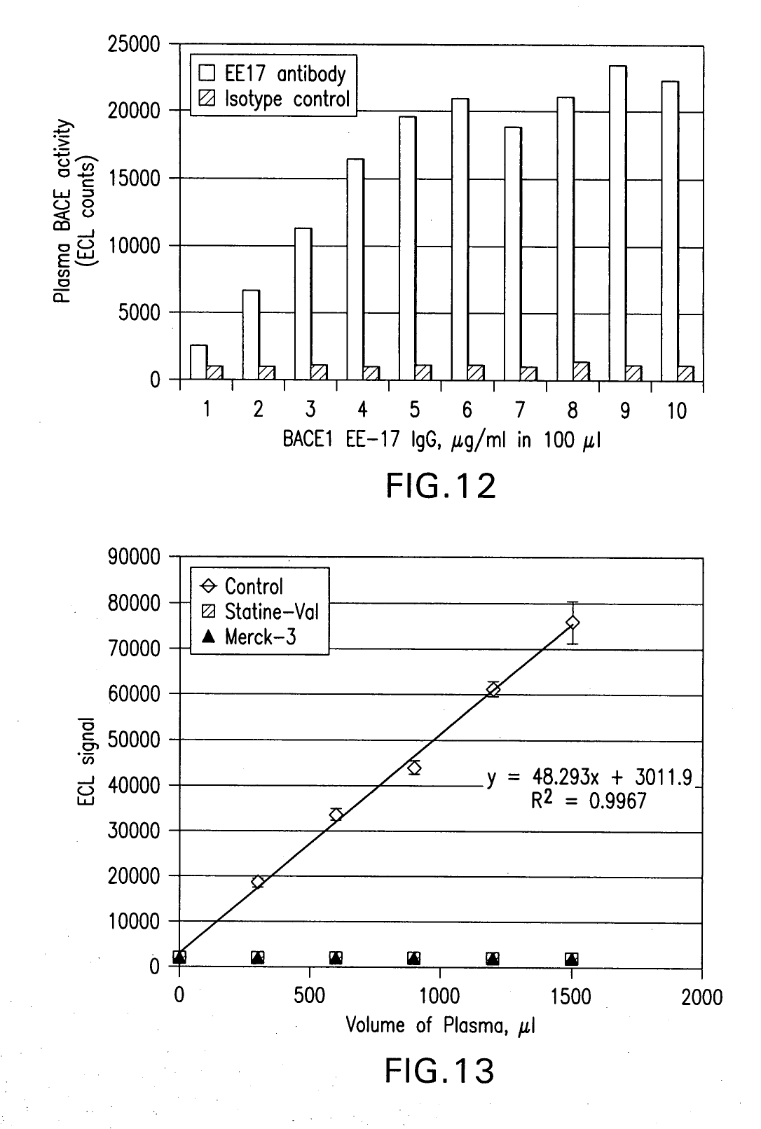

Following the above protocol, the inventors found that BACE-1 specific activity can be detected in serum samples obtained from humans. The inventors also found that detection of plasma BACE-1 activity was a function of anti-BACE-1 antibody concentration and plasma volume. As shown in FIG. 12, there was a concentration dependent and saturable increase in plasma BACE-1 activity which was dependent on anti-BACE-1 antibody concentration. The Figure shows that as the amount of anti-NF antibody was increased from 1 to 10 μg/mL, the ECL signal increase and appeared to plateau at an antibody concentration of between about 5 to 6 μg/mL. FIG. 13 shows that BACE-1-specific activity detected in plasma increased as the plasma volume was increased and the BACE-1 activity was completely blocked by the specific BACE-1 inhibitors Statine-Val and Merck-3 at a concentration of 10 μM.

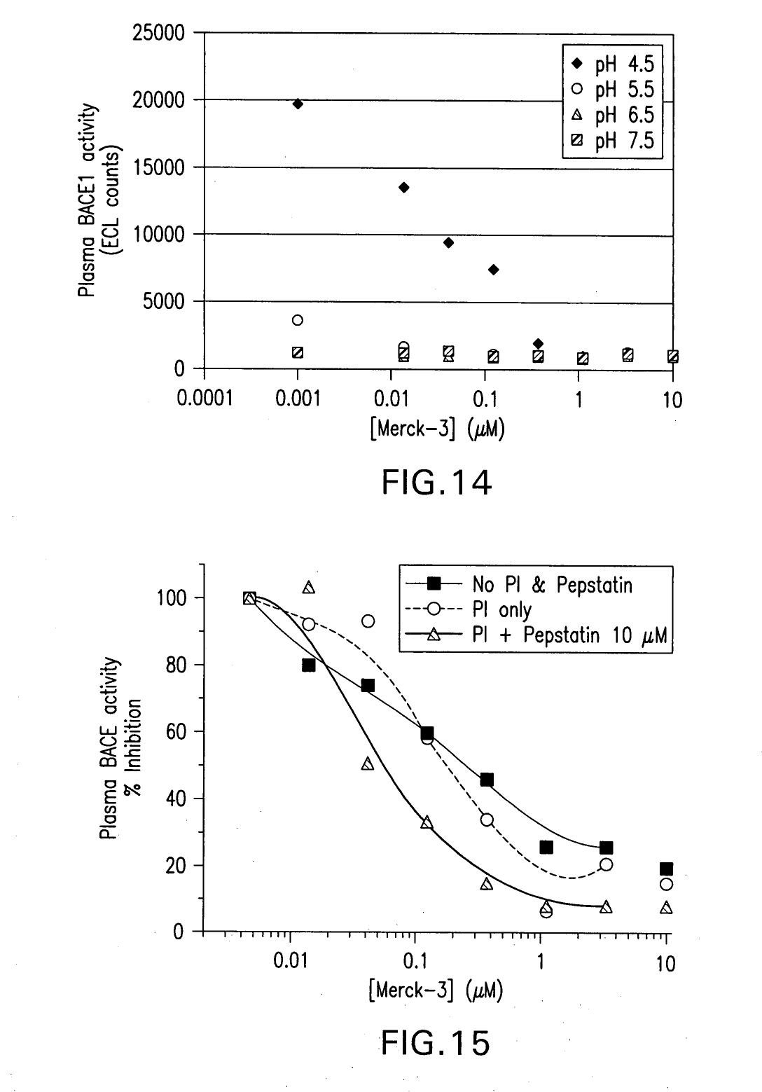

Detection of BACE-1 activity in plasma was dependent on the pH of the reaction mixture. FIG. 14 shows that detection of BACE-1 activity was highest at the acidic pH of about 4.5 and that as the pH was increased to pH 7.5, the ability to detect BACE-1 activity decreased. BACE-1 activity was undetectable when the pH was increased to 6.5 or 7.5. The BACE-1 activity at pH4.5 was abolished at Merck-3 concentrations greater than 1 μM.

FIG. 15 shows that a dose-dependent lowering of plasma BACE-1 activity was observed with the Merck-3 inhibitor, which had a have maximal inhibitory concentration (IC₅₀) of about 50 nM when the reaction mixture included both protease inhibitors and pepstatin A.

This example shows that BACE-1 activity can be detected in blood serum and plasma.

### EXAMPLE 4

This example demonstrates that the BACE-1 activity found in CSF samples is due to a carboxy terminal truncated BACE-1. Immunocapture was used to capture any BACE-1 in the CSF, which was then assayed for BACE-1 activity.

Protein A coated plates (Pierce Chemical CO., Cat #15130) are bound with the following BACE-1 polyclonal antibodies at a concentration of 2 μg/mL and incubated at room temperature for 2 hours: anti-BACE-1 antibody EE-17 (Sigma-Aldrich, Cat# B 0681; antibody specific for the epitope comprising amino acids 46 to 62), polyclonal anti-BACE-1 antibody (Chemicon, Cat #5832; antibody specific for an epitope within amino acids 458 to 501), and anti-BACE-1 antibody LK-16 (Sigma-Aldrich, Cat# B 0806; antibody specific for an epitope within amino acids 485 to 501). Negative control normal rabbit IgG (Sigma Cat# I 5006) was coated at the same concentration. 100 μL of Rhesus CSF is added to the wells and the plates incubated overnight. Baculovirus expressed truncated BACE-1 consisting of amino acids 1 to 460 (BBACE (aa460); same as baculo-BACE described in Examples 2 and 3) and membrane bound full length BACE-1 isolated from cell line expressing human BACE-1 are used as controls. The plates are then washed with PBS. The BACE-1 enzymatic cleavage reaction and the product of the BACE-1 enzymatic cleavage are measured by ELISA as described for the CSF BACE-1 activity assay described in Example 2.

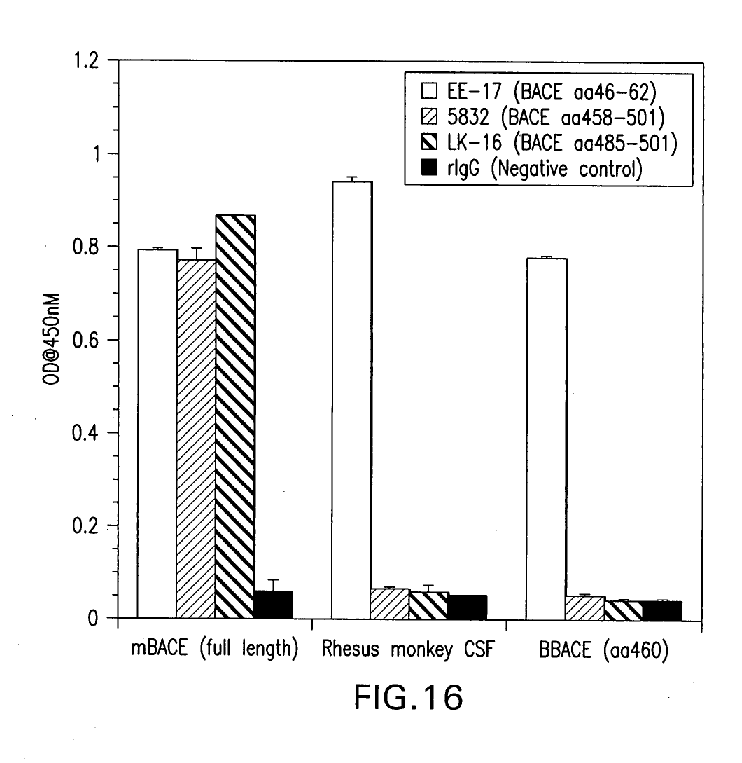

FIG. 16 shows that following the above method, BACE-1 activity was present in those wells that contained the amino terminal capture antibody EE-17, which was similar to the BACE-1 activity seen for those wells that contained EE-17 captured BBACE (aa460) and full-length membrane BACE-1 (mBACE). However, no BACE-1 activity for the CSF extract or BBACE (aa460) was seen in those wells that contained either carboxy terminal capture antibody. BACE-1 activity for the full-length BACE-1 was seen for all wells. The results indicate that BACE-1 in CSF exists as a carboxy terminal truncated protein missing at least amino acids 461- to 501.

To determine the molecular weight of the truncated BACE in Rhesus CSF, the BACE-1 specific peptide inhibitor statine-val was used to capture BACE-1 from the rhesus CSF using an affinity precipitation method and the captured BACE-1 electrophoresed on a Tris-glycine gel. Statine-val is conjugated to pre-packed NHS-activated SEPHAROSE High Performance column (Amersham Biosciences (GE Healthcare), Piscataway, N.J.; Cat#17-0716-01) following standard protocols. Then, 100 μL of the statine-val conjugated SEPHAROSE beads are used to capture BACE-1 from 10 mL of rhesus CSF equilibrated to pH 4.5 and containing 10 μM pepstatin-A. After overnight incubation, the beads are washed thoroughly with reaction buffer at pH 4.5. Then the statine-val conjugated SEPHAROSE beads are suspended in 200 μL of SDS-PAGE loading buffer and boiled for 10 minutes to elute any bound BACE-1 from the beads. Then, about 40 μL of the eluant is loaded onto 10% Tris-glycine polyacrylamide gel (Invitrogen, Cat# EC6075BOX.). BBACE (aa460) and full-length membrane BACE-1 are used as controls. The gel was electrophoresed 100V for 1.5 hours and then transferred to a PVDF membrane. BACE-1 was detected using antibody EE-17 (Sigma-Aldrich; Cat# B 0681) and labeled antibody against the EE-17 antibody.

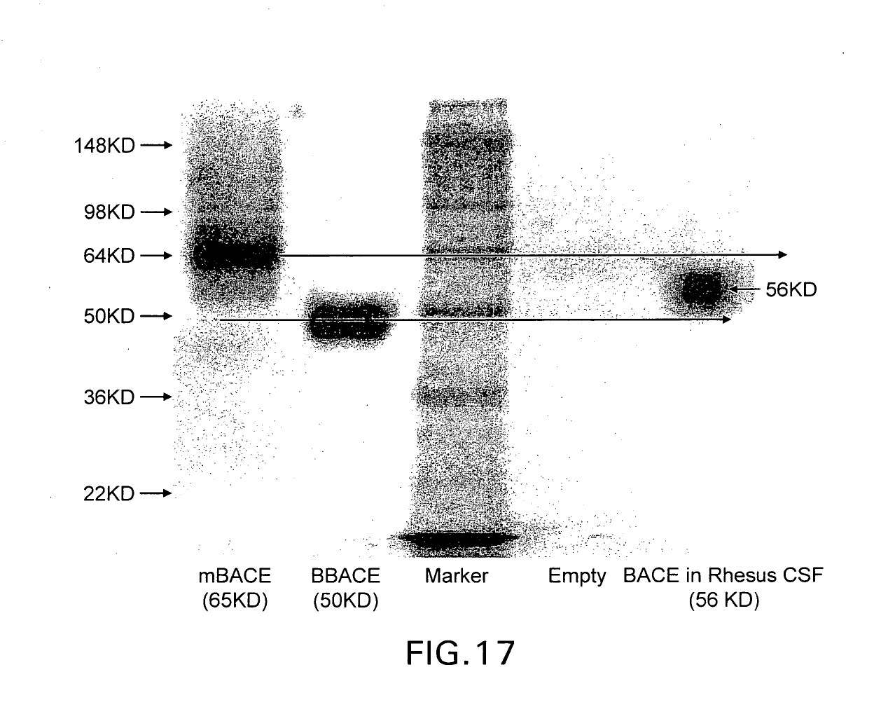

Following the above method, the molecular weight of the carboxy terminal truncated BACE-1 was determined to be between about 56 kDa. As shown in the Western blot shown in FIG. 17, full length membrane BACE-1 had a molecular weight of about 60 to 70 kDa while truncated BBACE (aa460) had a molecular weight of about 45 to 50 kDa. The results confirm that the BACE-1 in rhesus CSF exists as a c-terminal truncated form having a molecular weight of about 56 kDa. While the exact site of the carboxy terminal truncation was not defined to the amino acid level, the results show that the truncated BACE-1 might be somewhat longer than 460 amino acids.

### EXAMPLE 5

To confirm that the identity of the carboxy terminal truncated BACE-1 identified IN Example 4 using the immuno-capture assay and Western blot, the protein was isolated from an SDS polyacrylamide gel, digested with typsin to produce tryptic peptides, and the tryptic peptides analyzed by mass spectrometry.

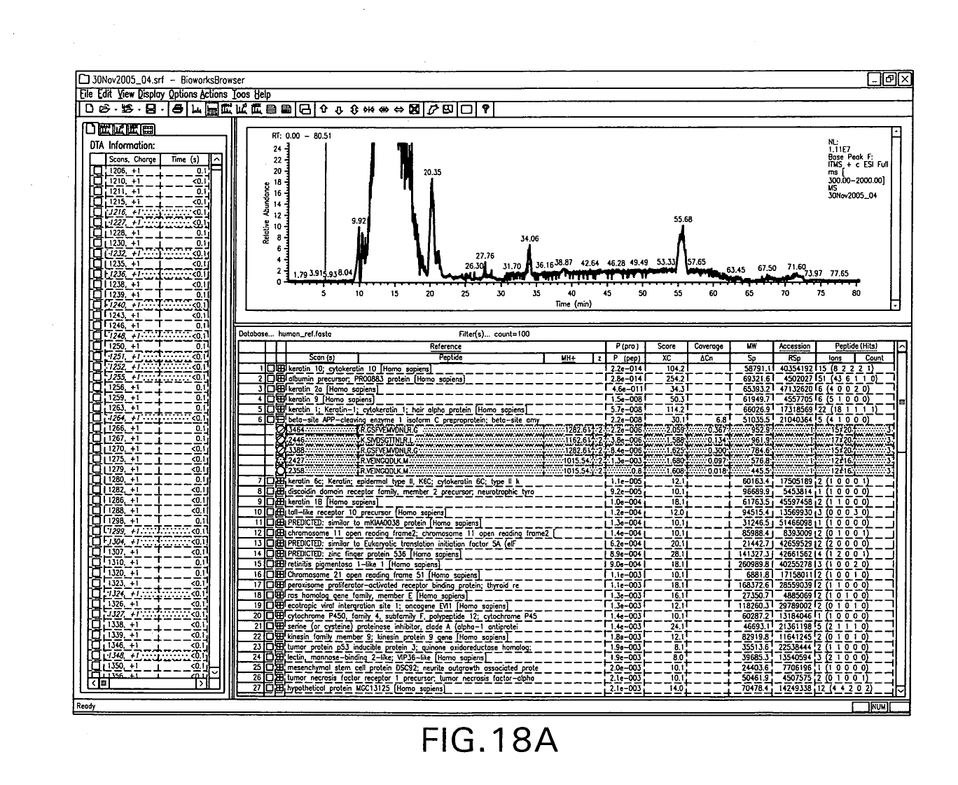

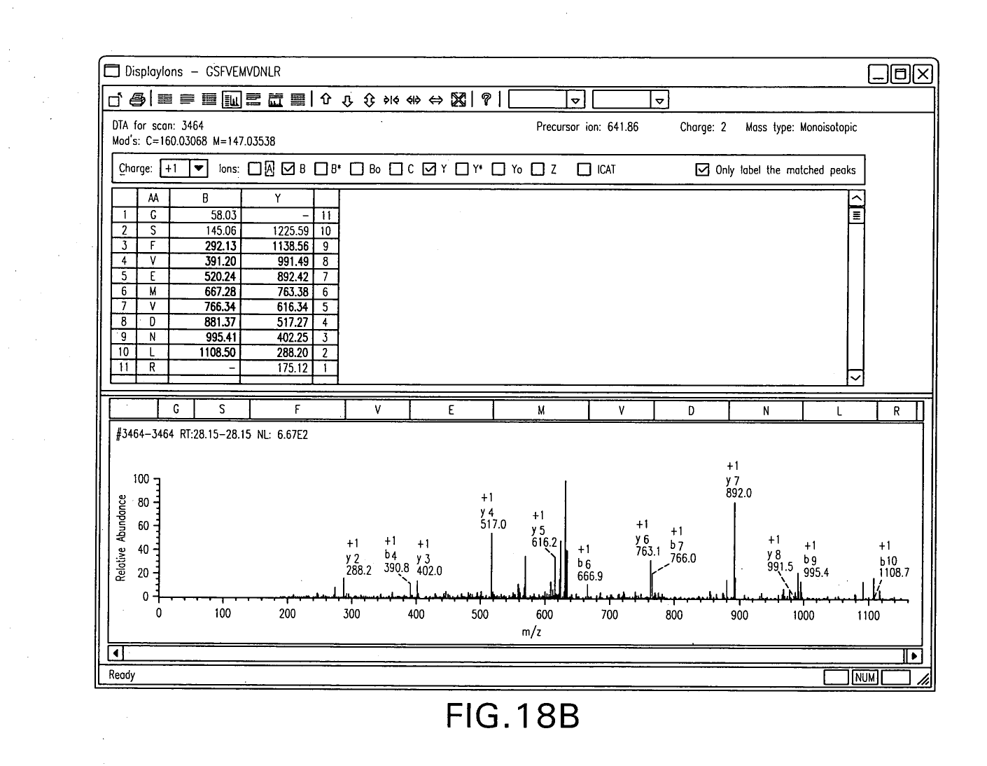

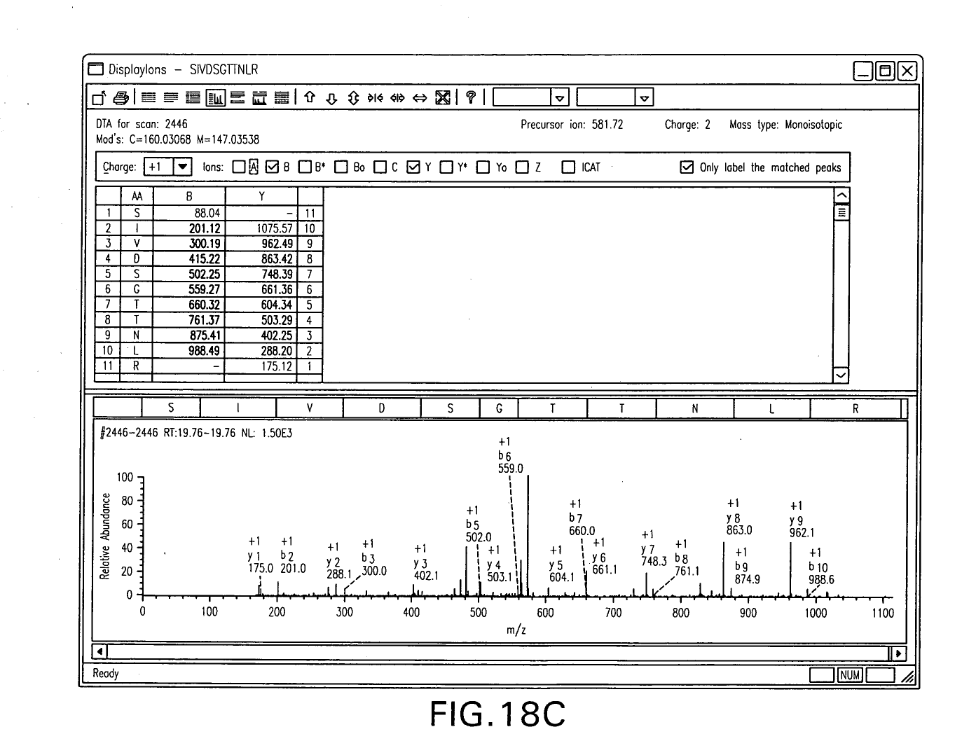

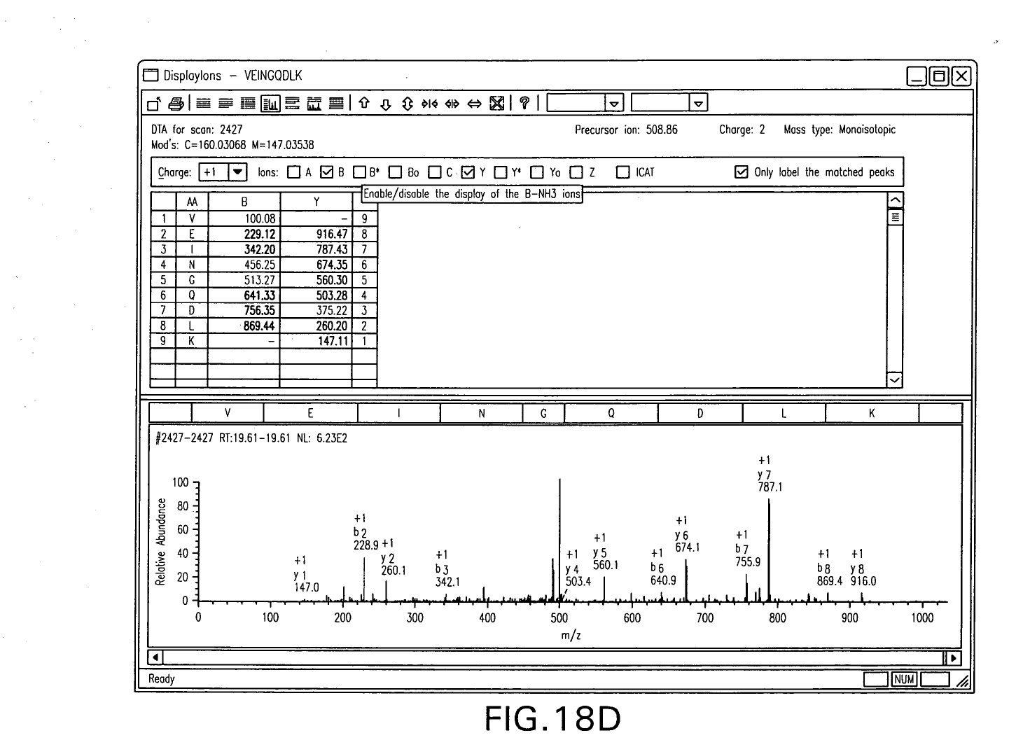

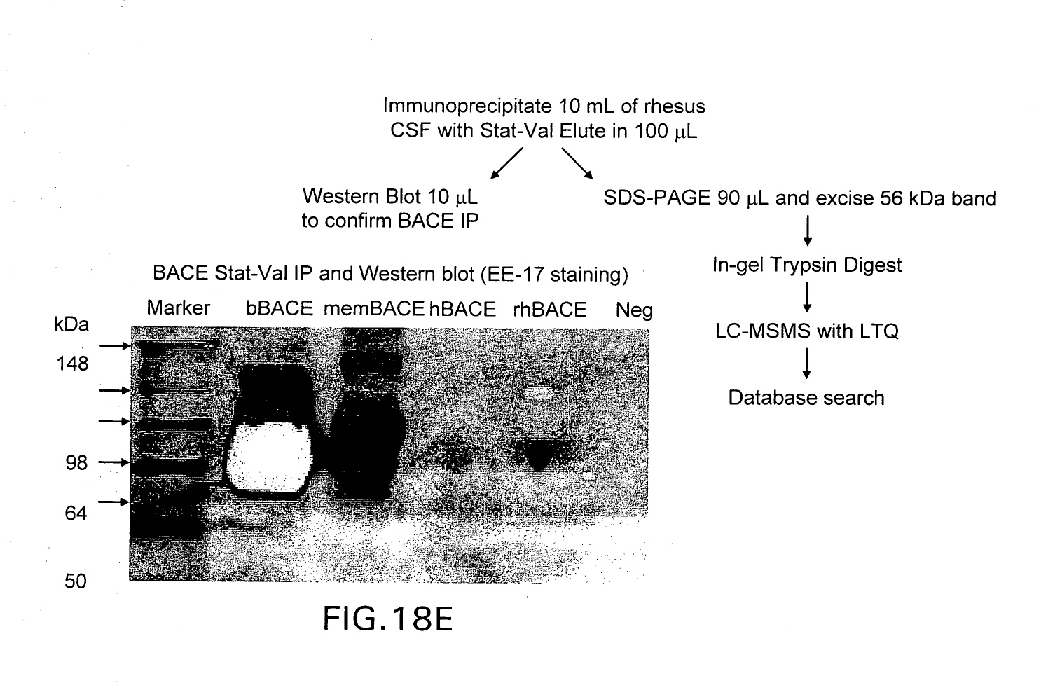

Ten mL of rhesus monkey CSF is subjected to the statine-val affinity precipitation method above. The bound carboxy truncated BACE-1 is eluted in 100 μL of SDS-PAGE loading buffer. About 90 μL of the eluant is subjected to electrophoresis on an SDS polyacrylamide gel. The remaining 10 μL is subjected to SDS gel electrophoresis to confirm the carboxy truncated BACE-1 is eluted from the gel. After electrophoresis, the 56 kDa band is cut from the gel and the protein therein digested in the gel with trypsin as follows. SDS gel slices are processed through a standard in-gel trypsin digestion protocol with dithiothreitol and iodoacetamide as the reduction and alkylation reagents, respectively. Promega sequencing grade trypsin can be used to reduce the occurrence of autolysis fragments. The digestion reaction is incubated at 37° C. for the first few hours and then 30° C. overnight. The reaction is quenched by the addition of acetic acid to reduce the pH to approximately 3. Afterwards, the tryptic peptides are separated from the gel and subjected to LC-MSMS (Liquid Chromatography Tandem Mass Spectrometry) with a Thermo-Electron LTQ ion trap mass spectrometer (Thermo-Electron Corporation, Waltham, Mass.) to sequence the peptides and the remaining 10 μL is subjected to SDS gel electrophoresis and Western blotting performed as described above to confirm that the carboxy terminal truncated BACE-1 had been eluted from the gel. The experimental scheme is shown in FIG. 18E.

Liquid chromatography mass spectrometry experiments are carried out using an LC Packings FAMOS autosampler (LC Packings, a Dionex Company, Sunnyvale, Calif.), Agilent 1100 Cap Pump (Agilent technologies Inc., Palo Alto, Calif.), and Thermo-Electron LTQ ion trap mass spectrometer (Thermo-Electron Corporation, Waltham, Mass.). Fairly standard single dimensional chromatography using a reversed phase trap and separation columns (0.1×25 mm PROTEOPEP II (New Objectives, Woburn, Mass.) and 0.1×5 mm POROS R2 (Perceptive Biosystems, Hertford, UK), respectively) at a flow rate of 1 μL/min can be used. The binary gradient elution includes a five minute wash step with Solvent A (0.5% acetic acid in water) and a 30 minute gradient at 1% per minute increase in Solvent B (90% acetonitrile, 10% Water, 0.5% acetic acid) followed by a 10 min ramp to 90% B to wash off residual peptide or protein. The LTQ mass spectrometer is setup to perform a single full mass spectrometry scan followed by 3 data dependent MSMS scans. The 3 data dependent MSMS scans are used to produce peptide specific sequence fragment ions that can be used in conjunction with the parent mass of the peptide to search a protein database.

Data obtained using the LC-MS methodology described above is used to search a database of human proteins for matches with BACE-1 specific tryptic fragments using TURBOSEQUEST (Thermo-Electron Corporation). TURBOSEQUEST is an XCALIBUR (Thermo-Electron Corporation) layered application that automatically identifies proteins by comparing experimental tandem mass spectrometry (MS/MS) data with standard protein and DNA databases. A human database can be used because a rhesus monkey database is not yet available and it is presumed that reasonable identity exists across these two closely related species such that BACE-1 tryptic peptides produced using rhesus monkey BACE-1 could be correlated to the human BACE-1 tryptic peptides in the database.

Following the above protocol above, the mass spectrometry data was compared to a database of predicted peptides for a library of proteins, including BACE-1 isoform C. BACE-1 isoform C has the same amino acid sequence as BACE-1 isoform A (SEQ ID NO:16) except for a 44 amino acid deletion from amino acid position 146 to amino acid position 189. FIG. 18A shows the mass spectrometry data and search results, which confirmed the identity of the band identified in the Western blot of FIG. 17 as BACE-1 based on identity of the three of the tryptic peptides that could be clearly sequenced. The Figure shows that of the tryptic peptides produced under the conditions used herein, three tryptic peptides of a tryptic digest of rhesus carboxy terminal truncated BACE-1 were unambiguously BACE-1 -derived tryptic peptides. FIG. 18B shows the parent peptide and m/z data for tryptic peptide from amino acid positions 58 to 68 of BACE-1 and consisting of amino acid sequence GSFVEMVDNLR (SEQ ID NO:13), FIG. 18C shows the parent peptide and m/z data for tryptic peptide from amino acid positions 86 to 94 of BACE-1 and consisting of amino acid sequence SIVDSGTTN. (SEQ ID NO:15), and FIG. 18D shows the parent peptide and m/z data for tryptic peptide from amino acid positions 67 to 75 of BACE-1 and consisting of amino acid sequence VEINGQDLK (SEQ ID NO:14), The results clearly show that the protein identified as a carboxy truncated BACE-1 is indeed derived from BACE-1.

### EXAMPLE 6

This prophetic example illustrates use of BACE-1 activity as a biomarker for Alzheimer's disease wherein the IC₅₀ of a serum sample from an individual measured against inhibitor Merck-3 is compared to the IC₅₀ for individuals with Alzheimer's disease and the IC₅₀ for individuals without Alzheimer's disease, both measured against Merck-3.

In the first step, the intrinisic IC₅₀ for serum from individuals who have Alzheimer's disease and for serum from individuals without Alzheimer's disease (normal individuals) is determined.

In the first step is the BACE-1 immuno-capture from plasma. About 100 μL of 8 μg/mL anti-BACE-1 antibody EE-17 (Sigma-Aldrich, Inc. Cat# B0681) is diluted in Binding Buffer (50 mM HEPES, 150 mM NaCl, 0.1% BSA, 0.1% Tween-20, pH 7.5) and added to each well of a REACTI-BIND Protein A coated plate (Pierce Chemical Co., Cat#15130). The plates are covered with plate sealers and then incubated at 4° C. over night with gentle shaking/rotating. After the incubation, the primary IgG is removed from the plate and 100 μL of 10 μg/mL rabbit IgG (Sigma-Aldrich, Inc., Cat# I-5006) diluted in Binding Buffer, pH 7.5 is added to each well of the plate to block all protein A sites. The plates are then incubated at room temperature for about 1.5 hours with shaking. The plates are then washed with 200 μL/well of PBS containing 0.05% Tween-20 three times and then washed with 200 μL/well of PBS two times. After washing, 300 μL aliquots of human serum or plasma from Alzheimer's individuals and normal individuals are separately added, one to each well to capture BACE-1 from the serum or plasma onto the plates. Typically, serum or plasma from 10 Alzheimer disease individuals and 10 normal individuals are used and multiple aliquots from each individual are used. The plates are covered with plate sealers and incubated at 4° C. over night with gentle shaking/rotating. After the incubation, the plates are washed with 200 μL/well PBS containing 0.05% Tween-20 two times, then washed with 200 μL per well PBS two times, and finally with 200 μL per well distilled H₂O two times.

The cleavage reaction is set up as follows. Each 100 μL/well reaction mixture contains 25 μL of 0.2 M Ammonium Acetate buffer, pH 4.5 (50 nM final); 10 μL of 10× Protease Inhibitor (1× final) (Roche Diagnostics GmbH, Cat#11836153001; provides 1.5 μg/mL chymotrypsin, 0.8 μg/mL thermolysin, 1 mg/mL papain, 1.5 μg/mL pronase, 1.5 μg/mL pancreatic extract, and 2 ng/mL trypsin); 10 μL of 1 mg/mL BSA (0.1 mg/mL final); 2 μL of 10% CHAPS (0.2% final) (Sigma-Aldrich, Inc., Cat#C-5070); 1 μL of 1 mM Pepstatin A (10 μM final) (Sigma-Aldrich, Inc., Cat# P-5318); 1 μL of DMSO or 1 μL of serial dilution of 1 mM Merck-3 to provide concentrations of Merck-3 ranging from about 0 μM to 10 μM; 1 μL of 25 μM N-terminus-labeled biotin-labeled 15-mer peptide substrate KTEEISEVNFEVEFR (SEQ ID NO:9) (250 nM final); and 50 μL distilled H₂O. After setting up the reactions, the plates are covered with plate sealers and incubated at 37° C. over night with gentle shaking/rotating. Then the reaction mix is transferred to a 96 well round-bottom polypropylene plate (Costar 3365).

Detection of BACE-1 activity for each aliquot can be by ECL as in Example 3 or ELISA as in Example 2. The Merck-3 IC₅₀ is then determined for each individual and then an Merck-3 intrinsic IC₅₀ determined for the Alzheimer's disease group and the normal group. The Merck-3 intrinsic IC₅₀ are used as standards to which is compared the Merck-3 IC₅₀ obtained from an individual being tested wherein the IC₅₀ is obtained as described above.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. A method for determining whether a biological sample has β-secretase (BACE-1) activity, which comprises:
(a) incubating the biological sample in a reaction mixture that includes a protease inhibitor that inhibits non-BACE-1 aspartyl proteases but not BACE-1 and a peptide substrate that includes the amino acid sequence NFEV (SEQ ID NO:3) as a cleavage site for BACE-1 for a time sufficient for any BACE-1 activity in the biological sample to cleave the peptide substrate at the BACE-1 cleavage site to produce a peptide product that has the amino acid sequence NF at the carboxy terminus or EV at the amino terminus;
(b) contacting the reaction mixture with an antibody that is specific for the amino acid sequence NF at the carboxy terminus of the peptide product; and
(c) detecting the antibody bound to the peptide product, wherein detection of the antibody bound to the peptide product indicates that the biological sample has BACE-1 activity.

2. The method of claim 1 wherein the peptide product has the amino acid sequence EV at the amino terminus.

3. The method of claim 1 wherein the peptide substrate comprises the amino acid sequence EVNFEVEF (SEQ ID NO:7).

4. The method of claim 1 wherein the peptide substrate comprises an amino acid sequence selected from the group consisting of KTEEISEVNFEVEFR (SEQ ID NO:8) and REVNFEVEFR (SEQ ID NO:9).

5. The method of claim 1 wherein the protease inhibitor is pepstatin A.

6. The method of claim 1 used for detecting Alzheimer's disease in an individual, which comprises:
(a) obtaining a biological fluid sample from the individual; and
(b) detecting the peptide product having the amino acid sequence NF at the carboxy terminus or EV at the amino terminus, wherein the presence of said peptide product indicates the individual has Alzheimer's disease.

7. The method of claim 1 used for determining the efficacy of a treatment for Alzheimer's disease in an individual, which comprises:
(a) obtaining a biological fluid sample from the individual before treatment and at various times during treatment;
(b) detecting the peptide product having the amino acid sequence NF at the carboxy terminus or EV at the amino terminus in the sample; and
(c) comparing the amount of said peptide product in the samples obtained at various times during treatment to the amount in the sample obtained before treatment to determine the efficacy of the treatment.

8. The method of claim 1 used for determining the efficacy of a drug candidate for treating Alzheimer's disease, which comprises:
(a) obtaining a biological fluid sample from an individual before treatment and at various times during treatment with the drug candidate;
(b) detecting the peptide product having the amino acid sequence NF at the carboxy terminus or EV at the amino terminus in the sample; and,
(c) comparing the amount of said peptide product in the samples obtained at various times during treatment to the amount in the sample before treatment to determine the efficacy of the drug candidate for treating Alzheimer's disease.

9. The method of claim 1 which further comprises:
(a) providing an antibody that binds BACE-1;
(b) incubating the antibody with the biological sample for a time sufficient for the antibody to bind any of the BACE-1 that might be in the biological sample; and
(c) separating the BACE-1 bound to the antibody from the biological sample, prior to incubating the separated BACE-1 bound to the antibody of step (c) in the reaction mixture that includes a protease inhibitor.

10. The method of claim 9 wherein the peptide product has the amino acid sequence EV at the amino terminus.

11. The method of claim 9 wherein the peptide substrate comprises the amino acid sequence EVNFEVEF (SEQ ID NO:7).

12. The method of claim 9 wherein the peptide substrate comprises an amino acid sequence selected from the group consisting of KTEEISEVNFEVEFR (SEQ ID NO:8) and REVNFEVEFR (SEQ ID NO:9).

13. The method of claim 9 wherein the protease inhibitor is pepstatin A.

14. A biomarker for Alzheimer's disease comprising a carboxy terminal truncated BACE-1, which has an apparent molecular weight of about 56 kDa.

15. A method for detecting Alzheimer's disease in an individual using the biomarker of claim 14, which comprises:
(a) obtaining a biological fluid sample from the individual; and
(b) detecting a carboxy terminal truncated BACE-1 having an apparent molecular weight of about 56 kDa, wherein the presence of said truncated BACE-1 indicates the individual has Alzheimer's disease.

16. A method used for determining the efficacy of a treatment for Alzheimer's disease in an individual using the biomarker of claim 14, which comprises:
(a) obtaining a biological fluid sample from the individual before treatment and at various times during treatment;
(b) detecting a carboxy terminal truncated BACE-1 having an apparent molecular weight of about 56 kDa in the sample; and
(c) comparing the amount of said truncated BACE-1 in the samples obtained at various times during treatment to the amount in the sample obtained before treatment to determine the efficacy of the treatment.

17. A method for determining the efficacy of a drug candidate for treating Alzheimer's disease using the biomarker of claim 14, which comprises:
(a) obtaining a biological fluid sample from an individual before treatment and at various times during treatment with the drug candidate;
(b) detecting a carboxy terminal truncated BACE-1 having an apparent molecular weight of about 56 kDa; and,
(c) comparing the amount of said truncated BACE-1 in the samples obtained at various times during treatment to the amount in the sample before treatment to determine the efficacy of the drug candidate for treating Alzheimer's disease.

18. A biomarker for Alzheimer disease comprising:
a biomarker having a 50% inhibitory concentration (IC₅₀) value for a BACE-1 inhibitor obtained from a biological sample from an individual which is greater than the IC₅₀ value for a BACE-1 inhibitor from a biological sample obtained from an individual that does not have Alzheimer's disease.

19. A kit for an assay for determining whether a biological sample has β-secretase (BACE-1) activity, which comprises:
(a) at least one protease inhibitor that inhibits non-BACE-1 aspartyl proteases;
(b) a peptide substrate that includes the amino acid sequence NFEV (SEQ ID NO:3) as a cleavage site for the BACE-1;
(c) an antibody that is specific for the amino acid sequence NF at the carboxy terminus or EV at the amino terminus of the peptide product produced when the peptide substrate is cleaved by the BACE-1; and
(d) instructions for performing the assay.


SEQUENCE LISTING (16 sequences)

SEQ ID NO: 1 - protein, 4 aa, Homo Sapien
  PEPTIDE (0)...(0): BACE-1 cleavage site
KMDA

SEQ ID NO: 2 - protein, 4 aa, Homo Sapien
  PEPTIDE (0)...(0): Swedish BACE-1 cleavage site
NLDA

SEQ ID NO: 3 - protein, 4 aa, Artificial Sequence
  mutant BACE-1 cleavage site
NFEV

SEQ ID NO: 4 - protein, 4 aa, Artificial Sequence
  mutant BACE-1 cleavage site
NFAA

SEQ ID NO: 5 - protein, 4 aa, Artificial Sequence
  mutant BACE-1 cleavage site
NFEA

SEQ ID NO: 6 - protein, 4 aa, Artificial Sequence
  mutant BACE-1 cleavage site
NFAV

SEQ ID NO: 7 - protein, 8 aa, Artificial Sequence
  peptide substrate
EVNFEVEF

SEQ ID NO: 8 - protein, 10 aa, Artificial Sequence
  peptide substrate
REVNFEVEFR

SEQ ID NO: 9 - protein, 15 aa, Artificial Sequence
  peptide substrate
KTEEISEVNFEVEFR

SEQ ID NO: 10 - protein, 5 aa, Artificial Sequence
  cleavage product
REVNF

SEQ ID NO: 11 - protein, 10 aa, Artificial Sequence
  cleavage product
KTEEISEVNF

SEQ ID NO: 12 - protein, 6 aa, Artificial Sequence
  antigence peptide
CSEVNF

SEQ ID NO: 13 - protein, 11 aa, Artificial Sequence
  tryptic peptide
GSFVEMVDNLR

SEQ ID NO: 14 - protein, 9 aa, Artificial Sequence
  tryptic peptide
SIVDSGTTN

SEQ ID NO: 15 - protein, 9 aa, Artificial Sequence
  tryptic peptide
VEINGQDLK

SEQ ID NO: 16 - protein, 10 aa, Homo Sapien
  PEPTIDE (0)...(0): BACE-1 cleavage site
SEVKMDAEFR